(19) United States
(12) Patent Application Publication
(10) Pub. No.: US 2022/0016267 A1
(43) Pub. Date: Jan. 20, 2022

(54) NANOPARTICLES FOR CANCER DETECTION

(71) Applicant: CITY OF HOPE, Duarte, CA (US)

(72) Inventors: Tom HABER, Pasadena, CA (US); Pengpeng CAO, Duarte, CA (US); Jacob BERLIN, Monrovia, CA (US); Karen ABOODY, Arcadia, CA (US)

(21) Appl. No.: 17/408,972

(22) Filed: Aug. 23, 2021

Related U.S. Application Data
(63) Continuation of application No. 16/066,626, filed on Jun. 27, 2018, now abandoned.
(60) Provisional application No. 62/274,028, filed on Dec. 31, 2015.

(51) Int. Cl. A61K 49/00

(52) U.S. Cl. CPC A61K 49/005; A61K 49/0093; A61K 49/0017

(57) ABSTRACT

Disclosed herein, inter alia, are methods for detecting cancer using nanoparticles.

### CROSS-REFERENCES TO RELATED APPLICATIONS

This application claims the benefit of U.S. Provisional Application No. 62/274,028, filed Dec. 31, 2015, which is incorporated herein by reference in entirety and for all purposes

### STATEMENT AS TO RIGHTS TO INVENTIONS MADE UNDER FEDERALLY SPONSORED RESEARCH AND DEVELOPMENT

This invention was made with government support under grant number RO1 CA197359 awarded by the National Institutes of Health. The government has certain rights in the invention.

### BACKGROUND

Improved imaging technologies enable earlier detection, and enhanced diagnosis, guidance, and evaluation of cancer therapies. Imaging tumors, especially small tumors, is critical for diagnosing cancer at an early or precancerous stage where surgical methods are the preferred form of treatment. Nanoparticles have been designed to act as contrasting agents or fluorescently labeled carriers to penetrate cells, but often the nanoparticles are not effective detection agents. Disclosed herein, inter alia, are solutions to these and other problems in the art.

### BRIEF SUMMARY OF THE INVENTION

In an aspect is provided a method of detecting a cancer cell or tumor in a subject including: (a) administering into the peritoneum of the subject a nanoparticle, wherein the nanoparticle includes a detectable agent; and (b) detecting the nanoparticle at the site of the cancer cell or the tumor in the subject; thereby detecting the cancer cell or tumor in the subject. In embodiments, the nanoparticle is an unmodified silica nanoparticle.

In another aspect is provided a nanoparticle-cell construct including an inorganic nanoparticle covalently attached to a protein through a covalent linker, the covalent linker having the formula: (Ia) L²-X¹-L¹-X²-L³- or (Ib) L²-X²-L³-; wherein X¹ and X² are independently a bioconjugate linker or a bond, wherein at least one of X¹ or X² is a bioconjugate linker; L¹ is independently a polymeric linker; L² is independently a bond, —NR^{1a}—, —O—, —S—, —C(O)—, —C(O)O—, —S(O)—, —S(O)₂—, —NR^{1a}C(O)—, —C(O)NR^{1b}—, —C(O)(CH₂)_{z1}—, —NR^{1a}C(O)O—, —NR^{1a}C(O)NR^{1b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L³ is independently a bond, —NR^{2a}—, —O—, —S—, —C(O)—, —C(O)O—, —S(O)—, —S(O)₂—, —NR^{2a}C(O)—, —C(O)NR^{2b}—, —C(O)(CH₂)_{z2}—, —NR^{2a}C(O)O—, —NR^{2a}C(O)NR^{2b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R^{1a}, R^{2a}, R^{1b}, and R^{2b} are independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and the symbols z1 and z2 are independently an integer from 1 to 10.

### BRIEF DESCRIPTION OF THE DRAWINGS

FIG. 1. The experimental synthesis scheme showing the bare silica nanoparticle, also referred to herein as an unmodified silica nanoparticle, and how it can be functionalized with functional groups (e.g., amines), or with polymers (e.g., polyethylene glycol).

FIGS. 2A-2B. Tumor detection by unmodified (i.e. terminated with hydroxyl groups) fluorescent silica nanoparticles in nude mice. EGFP-expressing OVCAR8 cells were injected IP and tumors developed on the surfaces of organs. Red-fluorescent silica NPs were injected IP and 4 days later organs were removed for imaging. FIG. 2A shows the signal from the EGFP. Tumors are shown with arrows showing area with signal in the green channel—wavelength: Em: 465 Ex: 510. FIG. 2B shows the unmodified silica nanoparticles in white (with arrows) imaged in the red channel Wavelength Em: 570, Ex: 610. The unmodified silica nanoparticles demonstrated good correlation in coverage with ovarian tumors.

FIGS. 3A-3B. Tumor detection by amine functionalized fluorescent silica nanoparticles in nude mice. EGFP-expressing OVCAR8 cells were injected IP and tumors developed on the surfaces of organs. Red-fluorescent amine functionalized silica NPs were injected IP and 4 days later organs were removed for imaging. FIG. 3A shows the signal from the EGFP. Tumors are shown with arrows showing area with signal in the green channel—wavelength: Em: 465 Ex: 510. FIG. 3B shows the red fluorescent signal from Amine-silica nanoparticles as white (with arrows) Wavelength Em: 570, Ex: 610. Very little correlation was seen between tumor signal and particle signal.

FIGS. 4A-4B. Tumor detection by PEG functionalized fluorescent silica nanoparticles in nude mice. EGFP-expressing OVCAR8 cells were injected IP and tumors developed on the surfaces of organs. Red-fluorescent PEG functionalized silica NPs were injected IP and 4 days later organs were removed for imaging. FIG. 3A shows the signal from the EGFP. Tumors are shown with arrows showing area with signal in the green channel—wavelength: Em: 465 Ex: 510. FIG. 4B shows PEG-silica nanoparticles are shown in white (with arrows) imaged in the red channel Wavelength Em: 570, Ex: 610. Moderate correlation was observed between tumor signal and particle signal

FIG. 5. The sensitivity of tumor detection by the different fluorescent silica nanoparticles. Sensitivity is defined as (the number of true positives)/(true positives+false positives), and refers to how many tumors the NP detected out of the total number of tumors.

FIG. 6. We sectioned the tumors and healthy organs and imaged them by a confocal microscope. FIG. 6 depicts an image, showing the NPs accumulate around the tumor (left) but not around the healthy liver (right). The white bar on the lower left measures 200 μM.

FIGS. 7A-7B. The kinetics of detection of the unmodified silica nanoparticle. We injected the unmodified silica NP to mice bearing ovarian tumors, euthanized and imaged the organs from the IP cavity after 1, 5, 24 hours and 4 days. The brightest spots in each image are positive areas. It shows that the detection of the nanoparticles has a time dependent mechanism (the signal is stronger in longer times, 4 days had the strongest signal). FIG. 7A shows the tumors—in white (green channel, left panel) and NP in white (red channel, right panel) from 2 mice in the 1 hr time point. FIG. 7B shows the tumors—in white (green channel, left panel) and NP in white (red channel, right panel) from 2 mice in the 4 days time point. The signal after 4 days is stronger than the rest of the time points taken. The hydroxyl-silica nanoparticles demonstrated good correlation in coverage with ovarian tumors after 4 days. The brightest spots in each image are positive areas. Images were taken by LEICA Z16 MACROSCOPE.

FIGS. 8A-8B. A comparison between two different routes of administration, intraperitoneal (IP) and intravenous (IV). We injected the unmodified-silica NP (i.e. hydroxy terminated) to mice bearing ovarian tumors (LP or IV), euthanized and imaged the organs from the intraperitoneal cavity after 4 days. FIG. 8A shows the tumors—in white (green channel, left panel) and NP in white (red channel, right panel) from 2 mice following IV administration. There are no NP detected in the red channel indicating no delivery via IV. FIG. 8B shows the tumors—in white (green channel, left panel) and NP in white (red channel, right panel) from 2 mice following IP administration. In the IP injections group there is a bright signal from the NP and it is demonstrate good correlation in coverage with ovarian tumors after 4 days.

FIGS. 9A-9D. Presented here are images of the green channel tumors. The surgery was done by looking at the red channel (NP) only. FIGS. 9A-9D show the reduction of the number of tumors after a surgery, the tumors are show in white (green channel) same mouse. FIG. 9A before surgery. FIG. 9B after the first part of the surgery (they cut everything they could see by a naked eye). FIG. 9C the second part of the surgery—after looking at the image of the red fluorescent silica NP (red channel) they cut everything they could detect. FIG. 9D the third part of the surgery—after looking again at the image of the red fluorescent silica NP (red channel) they cut everything they could detect. It is clear that by the silica NP we can detect more tumors than by naked eye. Images were taken by LEICA Z16 MACROSCOPE.

FIG. 10 shows the % reduction of the tumor area after surgery. There is higher reduction of tumor area after a surgery based on the detection of fluorescent SiNP than after a surgery based on the naked eye.

FIG. 11 shows human normal tissue (top) compared to tumor tissue (bottom) after being incubated with red fluorescent silica NP for different time points (1 hr, 1 day, 4 days and 1 week), and it can be seen that the NP accumulating in the tumor tissue with time (highest signal is after 4 days and then there is a plateau), while the NP do not accumulate in the normal tissue as in the tumor tissue. Images were taken by LEICA Z16 MACROSCOPE.

FIG. 12 shows human normal tissue compared to tumor tissue after being incubated with hydroxyl-red fluorescent silica NP and amine-red fluorescent silica NP for 4 days. It can be seen that the hydroxyl-red fluorescent silica NP have higher accumulation in the tumor tissue compared to normal tissue. There is no difference in the signal from the tumor and the normal tissue incubated with the amine-red fluorescent silica NP. Indicating that the hydroxyl-red fluorescent silica NP selectively accumulate in the tumor tissue compared to the normal tissue while the amine-red fluorescent silica NP have much lower accumulation in both tumor and normal tissues and with no selectivity between them. Images were taken by LEICA Z16 MACROSCOPE.

FIG. 13. Cartoon schema of a nanoparticle for use in MRI applications, showing an iron core surrounded by a hydroxyl-red fluorescent silica coating (e.g., silica conjugated to a fluorophore).

FIGS. 14A-14B. Tumor detection by Iron core@fluorescent silica shell nanoparticles in nude mice organs. Two last mice are control mice no NP were injected to these mice. FIG. 14A-14B shows the organs in the IP cavity of the same mice that were removed and imaged. FIG. 14A shows eGFP expressing ovarian cancer cells (OVCAR8) were injected IP to nude mice. Tumors are shown with arrows indicating positive signal in the green channel—wavelength: Em: 465 Ex: 510. FIG. 14B shows iron core@hydroxyl-silica shell nanoparticles are shown in white (with arrows) imaged in the red channel Wavelength Em: 570, Ex: 610. The iron core@hydroxyl-silica shell nanoparticles demonstrated good correlation in coverage with ovarian tumors. These may be used as contrast agents for MRI in order to detect tumors.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched non-cyclic chain, or combinations thereof, including at least one carbon atom and at least one heteroatom (e.g. O, N, P, Si, and S), and wherein the nitrogen and sulfur atoms may optionally be oxidized, and the nitrogen heteroatom may optionally be quaternized. The heteroatom(s) (e.g. O, N, P, S, and Si) may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH 2, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5(4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms (e.g., O, N, S, Si, or P). A heterocycloalkyl moiety may include three optionally different ring heteroatoms (e.g., O, N, S, Si, or P). A heterocycloalkyl moiety may include four optionally different ring heteroatoms (e.g., O, N, S, Si, or P). A heterocycloalkyl moiety may include five optionally different ring heteroatoms (e.g., O, N, S, Si, or P). A heterocycloalkyl moiety may include up to 8 optionally different ring heteroatoms (e.g., O, N, S, Si, or P).

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl”, “heteroalkyl”, “cycloalkyl”, “heterocycloalkyl”, “aryl”, and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, —NR′R″, —SR′, -halogen, —SiR′R″R′″, —OC(O)R′, —C(O)R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, S(O)₂R′, —S(O)₂NR′R″, —NRSO₂R′, NR′NR″R′″, ONR′R″, NR′C═(O)NR″NR′″R′″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, NR′NR″R′″, ONR′R″, NR′C═(O)NR″NR′″R′″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, independently substituted with at least one substituent selected from:
      (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, independently substituted with at least one substituent selected from: oxo,
         halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C_{10 aryl, and each substituted or unsubstituted heteroaryl is a substituted or unsubstituted} 5 to 9 membered heteroaryl.

In some embodiments, each substituted group described in the compositions herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compositions herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

The term “pharmaceutically acceptable salts” is meant to include salts of the active compositions that are prepared with relatively nontoxic acids or bases, depending on the particular substituents found on the compounds described herein. When compositions of the present invention contain relatively acidic functionalities, base addition salts can be obtained by contacting the neutral form of such compositions with a sufficient amount of the desired base, either neat or in a suitable inert solvent. Examples of pharmaceutically acceptable base addition salts include sodium, potassium, calcium, ammonium, organic amino, or magnesium salt, or a similar salt. When compositions of the present invention contain relatively basic functionalities, acid addition salts can be obtained by contacting the neutral form of such compositions with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compositions of the present invention contain both basic and acidic functionalities that allow the compositions to be converted into either base or acid addition salts. Other pharmaceutically acceptable carriers known to those of skill in the art are suitable for the present invention. Salts tend to be more soluble in aqueous or other protonic solvents than are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compositions of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compositions differs from the various salt forms in certain physical properties, such as solubility in polar solvents.

Provided herein are agents (e.g. compositions, drugs, therapeutic agents) that may be in a prodrug form. Prodrugs of the compounds described herein are those compounds that readily undergo chemical changes under select physiological conditions to provide the final agents (e.g. compositions, drugs, therapeutic agents). Additionally, prodrugs can be converted to agents (e.g. compositions, drugs, therapeutic agents) by chemical or biochemical methods in an ex vivo environment. Prodrugs described herein include compounds that readily undergo chemical changes under select physiological conditions to provide agents (e.g. compositions, drugs, therapeutic agents) to a biological system (e.g. in a subject).

Certain compositions of the present invention can exist in unsolvated forms as well as solvated forms, including hydrated forms. In general, the solvated forms are equivalent to unsolvated forms and are encompassed within the scope of the present invention. Certain compositions of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compositions used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compositions of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compositions of the present invention may also contain unnatural proportions of atomic isotopes at one or more of the atoms that constitute such compositions. For example, the compositions may be radiolabeled with radioactive isotopes, such as for example tritium (³H), iodine-125 (¹²⁵I), or carbon-14 (¹⁴C). All isotopic variations of the compositions of the present invention, whether radioactive or not, are encompassed within the scope of the present invention.

The symbol denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Descriptions of compositions of the present invention are limited by principles of chemical bonding known to those skilled in the art. Accordingly, where a group may be substituted by one or more of a number of substituents, such substitutions are selected so as to comply with principles of chemical bonding and to give compositions which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compositions.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat hyperproliferative disorders, such as cancer (e.g. ovarian cancer, bladder cancer, head and neck cancer, brain cancer, breast cancer, lung cancer, cervical cancer, bone cancer, spinal cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, or prostate cancer). For example certain methods herein treat cancer by decreasing or reducing or preventing the occurrence, growth, metastasis, or progression of cancer or by decreasing or reducing or preventing a symptom of cancer. Symptoms of cancer (e.g., ovarian cancer, bladder cancer, head and neck cancer, brain cancer, breast cancer, lung cancer, cervical cancer, bone cancer, spinal cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, or prostate cancer) would be known or may be determined by a person of ordinary skill in the art. The term “treating” and conjugations thereof, include prevention of an injury, pathology, condition, or disease (e.g. preventing the development of one or more symptoms of cancer (e.g. ovarian cancer, bladder cancer, head and neck cancer, brain cancer, breast cancer, lung cancer, cervical cancer, bone cancer, spinal cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, or prostate cancer).

As used herein, the term “cancer” refers to all types of cancer, neoplasm or malignant tumors found in mammals, including leukemia, lymphoma, carcinomas and sarcomas. Exemplary cancers that may be treated with a compound, pharmaceutical composition, or method provided herein include ovarian cancer, lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma, cisplatin resistant lung cancer, carboplatin resistant lung cancer, platinum-based compound resistant lung cancer), glioblastoma multiforme, glioma, or melanoma. Additional examples include, cancer of the thyroid, endocrine system, brain, breast, cervix, colon, head & neck, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer. In embodiments “cancer” refers to a cancer resistant to an anti-cancer therapy (e.g. treatment with an anti-cancer agent).

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

An “effective amount” is an amount sufficient to accomplish a stated purpose (e.g. achieve the effect for which it is administered, treat a disease, reduce enzyme activity, increase enzyme activity, reduce protein function, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug or prodrug is an amount of a drug or prodrug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g. cancer, ovarian cancer, bladder cancer, head and neck cancer, brain cancer, breast cancer, lung cancer, cervical cancer, bone cancer, spinal cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, or prostate cancer) means that the disease is caused by (in whole or in part), or a symptom of the disease is caused by (in whole or in part) the substance or substance activity or function. As used herein, what is described as being associated with a disease, if a causative agent, could be a target for treatment of the disease. For example cancer may be treated with a composition (e.g. compound, composition, nanoparticle, or conjugate, all as described herein) effective for inhibiting DNA replication.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein. In some embodiments contacting includes allowing a compound described herein to interact with a stromal cell. In some embodiments contacting includes allowing a compound described herein to interact with an immune cell. In some embodiments contacting includes allowing a compound described herein to interact with a protein associate with a stromal cell. In some embodiments contacting includes allowing a compound described herein to interact with a protein associated with an immune cell. In some embodiments contacting includes allowing a compound described herein to interact with the extracellular matrix generated by a stromal cell. In some embodiments contacting includes allowing a compound described herein to interact with the extracellular matrix generated by an immune cell.

As defined herein, the term “inhibition”, “inhibit”, “inhibiting” and the like in reference to a protein-inhibitor (e.g. antagonist) interaction means negatively affecting (e.g. decreasing) the level of activity or function of the protein relative to the level of activity or function of the protein in the absence of the inhibitor. In embodiments, inhibition refers to a decrease in DNA replication or transcription. In some embodiments inhibition refers to reduction of a disease or symptoms of disease (e.g. cancer, ovarian cancer, bladder cancer, head and neck cancer, brain cancer, breast cancer, lung cancer, cervical cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, or prostate cancer). Thus, inhibition may include, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein relative to the activity or function of the protein in the absence of the activator (e.g. compound described herein). Thus, activation may include, at least in part, partially or totally increasing stimulation, increasing or enabling activation, or activating, sensitizing, or up-regulating signal transduction or enzymatic activity or the amount of a protein decreased in a disease. Activation may include, at least in part, partially or totally increasing stimulation, increasing or enabling activation, or activating, sensitizing, or up-regulating signal transduction or enzymatic activity or the amount of a protein.

The term “modulator” refers to a composition that increases or decreases the level of a target molecule or the function of a target molecule.

“Patient” or “subject in need thereof” or “subject” refers to a living organism suffering from or prone to a disease or condition that can be treated by administration of a compound or pharmaceutical composition or by a method, as provided herein. Non-limiting examples include humans, other mammals, bovines, rats, mice, dogs, monkeys, goat, sheep, cows, deer, and other non-mammalian animals. In some embodiments, a patient is human. In some embodiments, a subject is human.

“Disease” or “condition” refer to a state of being or health status of a patient or subject capable of being treated with a compound, pharmaceutical composition, or method provided herein. In embodiments, the disease is cancer. In embodiments, the disease is cancer, ovarian cancer, bladder cancer, head and neck cancer, brain cancer, breast cancer, lung cancer, cervical cancer, bone cancer, spinal cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, or prostate cancer.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compositions with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation, to increase degradation of a prodrug and release of the drug, detectable agent). The compositions of the present invention can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Pharmaceutical compositions provided by the present invention include compositions wherein the active ingredient (e.g. compositions described herein, compounds described herein, including embodiments or examples) may be contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., reducing, eliminating, or slowing the progression of disease symptoms. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compositions described herein, the therapeutically effective amount can be initially determined from cell culture assays. Target concentrations will be those concentrations of active composition that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compositions effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compositions being employed. The dose administered to a patient, in the context of the present invention should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compositions effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compositions by considering factors such as compound potency, relative bioavailability, patient body weight, presence and severity of adverse side effects, preferred mode of administration and the toxicity profile of the selected agent.

The compositions described herein can be used in combination with one another, with other active agents (e.g. anti-cancer agents) known to be useful in treating a disease described herein (e.g. ovarian cancer, bladder cancer, head and neck cancer, brain cancer, breast cancer, lung cancer, cervical cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, or prostate cancer), or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent (e.g. anti-cancer agent). Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin (including recombinant interleukin II, or r1L.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), Vincristine sulfate, Cryptophycin 52 (i.e. LY-355703), Vitilevuamide, Tubulysin A, Canadensol, Centaureidin (i.e. NSC-106969), Oncocidin Al (i.e. BTO-956 and DIME), Fijianolide B, Laulimalide, Narcosine (also known as NSC-5366), Nascapine, Hemiasterlin, Vanadocene acetylacetonate, Monsatrol, lnanocine (i.e. NSC-698666), Eleutherobins (such as Desmethyleleutherobin, Desaetyleleutherobin, lsoeleutherobin A, and Z-Eleutherobin), Caribaeoside, Caribaeolin, Halichondrin B, Diazonamide A, Taccalonolide A, Diozostatin, (−)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Gurin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, or the like.

“Analog” and “analogue” are used interchangeably and are used in accordance with their plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof. Accordingly, an analog is a compound that is similar or comparable in function and appearance but not in structure or origin to a reference compound.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature. Sulfur-containing amino acids refers to naturally occurring and synthetic amino acids comprising sulfur, e.g., methionine, cysteine, homocysteine, and taurine.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “bioconjugate” or “bioconjugate linker” refers to the resulting association between atoms or molecules of bioconjugate reactive groups. The association can be direct or indirect. For example, a conjugate between a first bioconjugate reactive group (e.g., —NH₂, —COOH, —N-hydroxysuccinimide, or maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., sulfo-N-hydroxysuccinimide moiety) is covalently attached to the second bioconjugate reactive group (e.g. an amine). The term “haloacetyl,” as used herein, refers to a functional group having the formula:

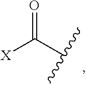

wherein X is a halogen.

Useful bioconjugate reactive groups used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(O) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

A “nanoparticle,” as used herein, is a particle wherein the longest diameter is less than or equal to 1000 nanometers. Nanoparticles may be composed of any appropriate material. For example, nanoparticle cores may include appropriate metals and metal oxides thereof (e.g., a metal nanoparticle core), carbon (e.g., an organic nanoparticle core) silicon and oxides thereof (e.g., a silicon nanoparticle core) or boron and oxides thereof (e.g., a boron nanoparticle core), or mixtures thereof. Nanoparticles may be composed of at least two distinct materials, one material (e.g., iron oxide) forms the core and the other material forms the shell (e.g., silica) surrounding the core.

An “inorganic nanoparticle” refers to a nanoparticle without carbon. For example, an inorganic nanoparticle may refer to a metal or metal oxide thereof (e.g., gold nanoparticle, iron nanoparticle) silicon and oxides thereof (e.g., a silica nanoparticle), or titanium and oxides thereof (e.g., titanium dioxide nanoparticle). In embodiments, the inorganic nanoparticle is a silica nanoparticle. The inorganic nanoparticle may be a metal nanoparticle. When the nanoparticle is a metal, the metal may be titanium, zirconium, gold, silver, platinum, cerium, arsenic, iron, aluminum or silicon. The metal nanoparticle may be titanium, zirconium, gold, silver, or platinum and appropriate metal oxides thereof. In embodiments, the nanoparticle is titanium oxide, zirconium oxide, cerium oxide, arsenic oxide, iron oxide, aluminum oxide, or silicon oxide. The metal oxide nanoparticle may be titanium oxide or zirconium oxide. The nanoparticle may be titanium. The nanoparticle may be gold. In embodiments, the metal nanoparticle is a gold nanoparticle. In embodiments, the inorganic nanoparticle may further include a moiety which contains carbon (e.g., fluorophore).

The term “silica nanoparticle” is used according to its plain and ordinary meaning and refers to a nanoparticle containing Si atoms (e.g., in a tetrahedral coordination) with 4 oxygen atoms surrounding a central Si atom. A person of ordinary skill in the art would recognize that the silica nanoparticle typically includes terminal oxygen atoms (e.g., the oxygens on the surface of the nanoparticle) that are hydroxyl moieties. A silica nanoparticle is a particle wherein the longest diameter is typically less than or equal to 1000 nanometers comprising a matrix of silicon-oxygen bonds. In embodiments, a nanoparticle has a shortest diameter greater than or equal to 1 nanometer (e.g., diameter from 1 to 1000 nanometers). In embodiments, the silica nanoparticle is mesoporous. In embodiments, the silica nanoparticle is nonporous.

A functionalized silica nanoparticle, as used herein, may refer to the post hoc conjugation (i.e. conjugation after the formation of the silica nanoparticle) of a moiety to the hydroxyl surface of a nanoparticle. For example, a silica nanoparticle may be further functionalized to include additional atoms (e.g., nitrogen) or chemical entities (e.g., polymeric moieties or bioconjugate group). For example, when the silica nanoparticle is further functionalized with a nitrogen containing compound, one of the surface oxygen atoms surrounding the Si atom may be replaced with a nitrogen containing moiety.

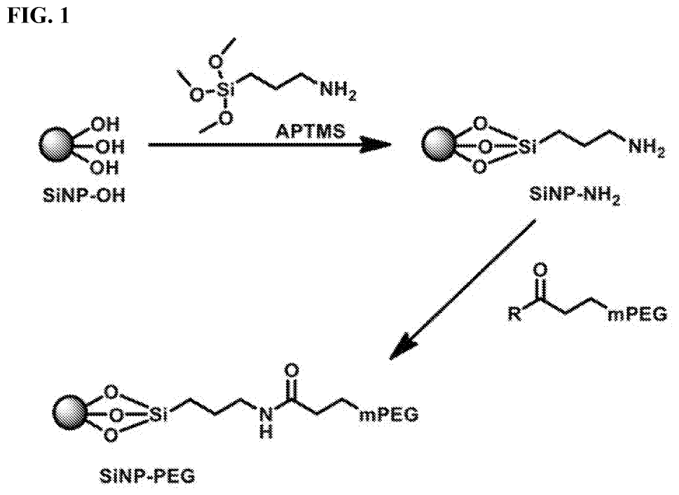

In contrast to a functionalized silica nanoparticle, an unmodified silica nanoparticle refers to a silica nanoparticle which has not been further functionalized, see FIG. 1. Thus, for example, an unmodified silica nanoparticle does not include a nitrogen containing moiety (e.g., terminal amine moieties). For example, an unmodified silica nanoparticle refers to a silica nanoparticle as synthesized without post hoc functionalization. Thus, in embodiments, the unmodified silica nanoparticles includes the following example

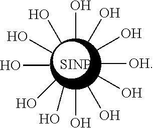

As used herein, the terms “bare silica nanoparticle” and “unmodified silica nanoparticle” are synonymous and interchangeable. In embodiments, an unmodified silica nanoparticle includes a detectable agent (e.g., fluorophore or stabilizer) which is incorporated (e.g., covalently or non-covalently) to the nanoparticle.

A “detectable agent” or “detectable compound” is a composition detectable by spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition (e.g., a nanoparticle or silica nanoparticle).

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²CU, ⁶⁴CU, ⁶⁷CU, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³B, ²²³Ra and ²²⁵AC. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Examples of detectable agents include imaging agents, including fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phoshatase, glucose oxidase, β-galactosidase, β-glucoronidase or β-lactamase. Such enzymes may be used in combination with a chromogen, a fluorogenic compound or a luminogenic compound to generate a detectable signal.

The term “polymeric” refers to a molecule including repeating subunits (e.g., polymerized monomers). For example, polymeric molecules may be based upon polyethylene glycol (PEG), poly[amino(1-oxo-1,6-hexanediyl)], or poly(oxy-1,2-ethanediyloxycarbonyl-1,4-phenylenecarbonyl). See, for example, “Chemistry of Protein Conjugation and Cross-Linking” Shan S. Wong CRC Press, Boca Raton, Fla., USA, 1993; “BioConjugate Techniques” Greg T. Hermanson Academic Press, San Diego, Calif., USA, 1996; “Catalog of Polyethylene Glycol and Derivatives for Advanced PEGylation, 2004” Nektar Therapeutics Inc, Huntsville, Ala., USA, which are incorporated by reference in their entirety for all purposes. In embodiments, the polymeric linker is divalent PEG. In embodiments, the polymeric moiety is monovalent PEG.

The term “polymerizable monomer” is used in accordance with its meaning in the art of polymer chemistry and refers to a compound that may covalently bind chemically to other monomer molecules (such as other polymerizable monomers that are the same or different) to form a polymer.

The term “branched polymer” is used in accordance with its meaning in the art of polymer chemistry and refers to a molecule including repeating subunits, wherein at least one repeating subunit (e.g., polymerizable monomer) is covalently bound to a different subunit (e.g., polymerizable monomer). For example a branched polymer has the formula:

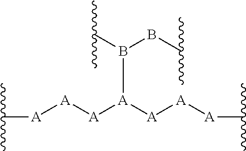

wherein ‘A’ is the first repeating subunit and ‘13’ is the second repeating subunit. In embodiments, the first repeating subunit (e.g., polyethylene glycol) is optionally different than the second repeating subunit (e.g., polymethylene glycol).

As used herein, the term “stabilizing agent” refers to a substance that aids the incorporation of a detectable agent to a nanoparticle and can be included in the compositions of the present invention without diminishing detectability. In some embodiments, the stabilizing agent may be an amino acid. In some embodiments, the stabilizing agent may be a charged polymer (e.g. cationic polymer), polysaccharide, polyelectrolyte, polyacid, polymer, dextran, polaxamer, surfactant, a glycerol, an erythritol, an arabinose, a xylose, a ribose, an inositol, a fructose, a galactose, a maltose, a glucose, a mannose, a trehalose, a sucrose, a polyethylene glycol, a carbomer 1342, a glucose polymers, a silicone polymer, a polydimethylsiloxane, a polyethylene glycol, a carboxy methyl cellulose, a poly(glycolic acid), a poly(lactic-co-glycolic acid), a polylactic acid, a dextran, poloxamers, organic co-solvents selected from ethanol, N-methyl-2-pyrrolidone (NMP), PEG 300, PEG 400, PEG 200, PEG 3350, Propylene Glycol, N,N Dimethylacetamide, dimethyl sulfoxide, solketal, tetahydrofurfuryl alcohol, diglyme, ethyl lactate, a salt (e.g. NaCl), a buffer or a combination thereof.

The term “macrophage” is used in accordance with its ordinary meaning and refers to a cell which is capable of phagocytosis. Due to differences in receptor expression, cytokine production, and functions, a macrophage may be referred to as Type I or Type II. Type I macrophages are cells capable of producing pro-inflammatory cytokines and are implicated in the killing of pathogens and tumor cells. Type II macrophages moderate the inflammatory response, eliminate cell wastes, and promote angiogenesis and tissue remodeling.

### II. Compounds

Provided herein are nanoparticles that are, inter alia, useful for the detection of cells. The nanoparticle may be an inorganic nanoparticle. In embodiments, the nanoparticle is a silica nanoparticle. The inorganic nanoparticle may be a metal nanoparticle. When the nanoparticle is a metal, the metal may be titanium, zirconium, gold, silver, platinum, cerium, arsenic, iron, aluminum, silicon, or mixtures thereof. The metal nanoparticle may be titanium, zirconium, gold, silver, or platinum and appropriate metal oxides thereof. In embodiments, the nanoparticle is titanium oxide, zirconium oxide, cerium oxide, arsenic oxide, iron oxide, aluminum oxide, or silicon oxide. The metal oxide nanoparticle may be titanium oxide or zirconium oxide. The nanoparticle may be titanium. The nanoparticle may be gold.

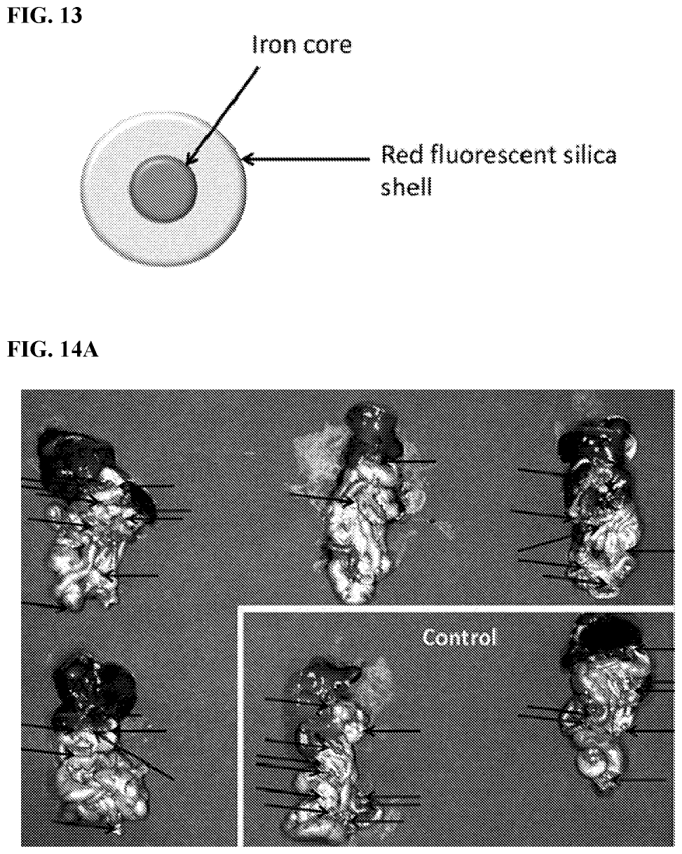

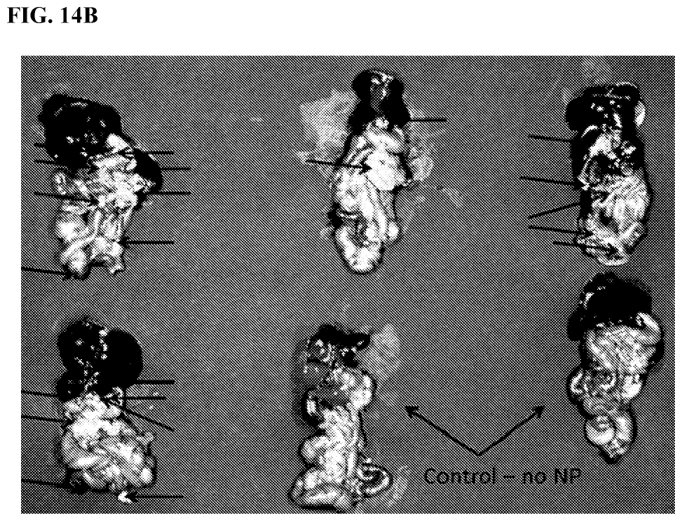

In embodiments, the nanoparticle includes two homogeneous materials (e.g., metal core and silica shell), see FIG. 13. The term silica shell refers to a coating containing Si atoms (e.g., in a tetrahedral coordination) with 4 oxygen atoms surrounding a central Si atom which surrounds a core. The core of the nanoparticle may contain a metal or oxide thereof. The metal-containing cores may be magnetic, paramagnetic or superparamagnetic. The metal of the metal-containing cores may be iron (e.g., Fe₃O₄ or Fe₂O₃), magnesium, cobalt, or mixtures thereof. In embodiments, the nanoparticle is an iron oxide core (e.g., magnetite or maghemite) with a silica shell. The silica shell may surround at least a portion of the core. The longest diameter of the core is from about 10 nm to about 500 nm. The thickness of the shell may range from about 0.01 nm to about 500 nm. The silica shell may cover a portion of the core nanoparticle. In embodiments, the silica shell covers about 1 to about 100% of the core. In embodiments, the silica shell covers about 10 to about 80% of the core. In embodiments the silica shell further includes a detectable agent. In embodiments, the silica shell covers about 10%, 20%, 30%, 40%, 50%, 60%, 70%, 80%, 90%, or about 100% of the core.

In embodiments, the silica nanoparticle is an unmodified silica nanoparticle. In embodiments, the silica nanoparticle is a non-polymeric functionalized silica nanoparticle (i.e. a silica nanoparticle that does not include polymers conjugated to the surface of the silica nanoparticle). In embodiments, the silica nanoparticle is a non-pegylated functionalized silica nanoparticle (i.e. a silica nanoparticle that does not include PEG polymers conjugated to the surface of the silica nanoparticle). In embodiments, the silica nanoparticle is a non-functionalized silica nanoparticle (i.e. a silica nanoparticle that does not include reactive chemical functional groups, such as a bioconjugate reactive group, conjugated to the surface of the silica nanoparticle (other than the terminal hydroxyl groups).

In embodiments, the unmodified silica nanoparticle includes terminal oxygen atoms (e.g., the oxygens on the surface of the nanoparticle) that are hydroxyl moieties. In embodiments, the terminal oxygen atoms of the unmodified silica nanoparticle are —OH or salts thereof (e.g. —O⁻ moieties). In embodiments, the terminal oxygen atoms of the unmodified silica nanoparticle may include an —OR″ moiety, wherein R″ is a substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl or substituted (e.g., substituted with a substituent group, a size-limited substituent group, or lower substituent group) or unsubstituted heteroaryl. In embodiments, about 70%, 80%, 90%, 95%, 99%, or about 100% of the terminal oxygen atoms of the unmodified silica nanoparticle are hydroxyl moieties (or salts thereof). In embodiments, about 70%, 80%, 90%, 95%, 99%, or about 100% of the terminal oxygen atoms of the unmodified silica nanoparticle are hydroxyl moieties (or salts thereof). In embodiments, about 90%, 91%, 92%, 93%, 94%, 95%, 96%, 97%, 98%, 99%, 99.9%, or about 100% of the terminal oxygen atoms of the unmodified silica nanoparticle are hydroxyl moieties (or salts thereof). In embodiments, the unmodified silica nanoparticle does not include a covalent bond to an additional chemical moiety (e.g., detectable agent or stabilizer). In embodiments, the unmodified silica nanoparticle includes a covalent bond to an additional chemical moiety (e.g., detectable agent or stabilizer). In embodiments, once the unmodified silica nanoparticle has formed, no further chemistry is performed to attach an additional chemical moiety (e.g., detectable agent or stabilizer) to the surface of the nanoparticle.

In embodiments, the detectable agent is incorporated (e.g., covalently or non-covalently) within the silica nanoparticle. In embodiments, the detectable agent is incorporated (e.g., covalently or non-covalently) throughout the silica nanoparticle (e.g., evenly distributed throughout the silica nanoparticle, distributed throughout the silica nanoparticle (e.g., in varying local concentrations, distributed within +/−10, 20, 30, 40, 50, 60, 70, 80, 90, or 100% of the average local concentration). In embodiments, the detectable agent (e.g., fluorophore) is distributed within about 10, 20, 30, 40, 50, 60, 70, 80, 90, or 100% of the average local concentration. In embodiments, the detectable agent (e.g., fluorophore) is conjugated to the surface and within the silica nanoparticle. In embodiments, the detectable agent (e.g., fluorophore) is at the surface of the silica nanoparticle (e.g., bonded covalently or non-covalently). In embodiments, the detectable agent is encapsulated within the silica nanoparticle (e.g., a detectable agent particle within the nanoparticle (e.g., a detectable agent particle of greater than 60, 70, 80, 90, 91, 92, 93, 94, 95, 96, 97, 98, or 99% detectable agent or a detectable agent particle of about 100% detectable agent)). In embodiments, the detectable agent is encapsulated within the silica nanoparticle and at the surface.

In embodiments, the silica nanoparticle is an unmodified silica nanoparticle. In embodiments, the silica nanoparticle is an unmodified silica nanoparticle which includes a detectable agent (e.g., fluorophore). In embodiments, the silica nanoparticle is an unmodified silica nanoparticle which includes a detectable agent (e.g., fluorophore).

In embodiments, the silica:detectable agent mass ratio is about 10:1 to 100:1. In embodiments, the silica:detectable agent mass ratio is about 10:1 to 90:1. In embodiments, the silica:detectable agent mass ratio is about 10:1 to 80:1. In embodiments, the silica:detectable agent mass ratio is about 10:1 to 70:1. In embodiments, the silica:detectable agent mass ratio is about 10:1 to 60:1. In embodiments, the silica:detectable agent mass ratio is about 10:1 to 50:1.

In embodiments, the silica:detectable agent mass ratio is about 10:1 to 40:1. In embodiments, the silica:detectable agent mass ratio is about 11:1. In embodiments, the silica:detectable agent mass ratio is about 12:1. In embodiments, the silica:detectable agent mass ratio is about 13:1. In embodiments, the silica:detectable agent mass ratio is about 14:1. In embodiments, the silica:detectable agent mass ratio is about 15:1. In embodiments, the silica:detectable agent mass ratio is about 16:1. In embodiments, the silica:detectable agent mass ratio is about 17:1. In embodiments, the silica:detectable agent mass ratio is about 18:1. In embodiments, the silica:detectable agent mass ratio is about 19:1. In embodiments, the silica:detectable agent mass ratio is about 20:1. In embodiments, the silica:detectable agent mass ratio is about 21:1. In embodiments, the silica:detectable agent mass ratio is about 22:1. In embodiments, the silica:detectable agent mass ratio is about 23:1. In embodiments, the silica:detectable agent mass ratio is about 24:1. In embodiments, the silica:detectable agent mass ratio is about 25:1. In embodiments, the silica:detectable agent mass ratio is about 26:1. In embodiments, the silica:detectable agent mass ratio is about 27:1. In embodiments, the silica:detectable agent mass ratio is about 28:1. In embodiments, the silica:detectable agent mass ratio is about 29:1. In embodiments, the silica:detectable agent mass ratio is about 30:1. In embodiments, the silica:detectable agent mass ratio is about 31:1. In embodiments, the silica:detectable agent mass ratio is about 32:1. In embodiments, the silica:detectable agent mass ratio is about 33:1. In embodiments, the silica:detectable agent mass ratio is about 34:1. In embodiments, the silica:detectable agent mass ratio is about 35:1. In embodiments, the silica:detectable agent mass ratio is about 36:1. In embodiments, the silica:detectable agent mass ratio is about 37:1. In embodiments, the silica:detectable agent mass ratio is about 38:1. In embodiments, the silica:detectable agent mass ratio is about 39:1. In embodiments, the silica:detectable agent mass ratio is about 40:1.

In embodiments, the average longest dimension of the nanoparticle is from about 10 nm to about 1000 nm. In embodiments, the average longest dimension of the nanoparticle is from about 10 nm to about 900 nm. In embodiments, the average longest dimension of the nanoparticle is from about 10 nm to about 800 nm. In embodiments, the average longest dimension of the nanoparticle is from about 10 nm to about 700 nm. In embodiments, the average longest dimension of the nanoparticle is from about 100 nm to about 400 nm. In embodiments, the average longest dimension of the nanoparticle is from about 200 nm to about 500 nm. In embodiments, the average longest dimension of the nanoparticle is from about 300 nm to about 500 nm. In embodiments, the average longest dimension of the nanoparticle is from about 500 nm to about 1000 nm. In embodiments, the average longest dimension of the nanoparticle is from about 400 nm to about 800 nm.

In embodiments, the average longest dimension of the nanoparticle is from about 10 nm to about 600 nm. In embodiments, the average longest dimension of the nanoparticle is from about 10 nm to about 300 nm. In embodiments, the average longest dimension of the nanoparticle is from about 10 nm to about 100 nm. In embodiments, the average longest dimension of the nanoparticle is from about 10 nm to about 90 nm. In embodiments, the average longest dimension of the nanoparticle is from about 10 nm to about 80 nm. In embodiments, the average longest dimension of the nanoparticle is from about 10 nm to about 70 nm. In embodiments, the average longest dimension of the nanoparticle is from about 10 nm to about 60 nm. In embodiments, the average longest dimension of the nanoparticle is from about 10 nm to about 50 nm. In embodiments, the average longest dimension of the nanoparticle is from about 25 nm to about 75 nm. In embodiments, the average longest dimension of the nanoparticle is from about 40 nm to about 60 nm. In embodiments, the average longest dimension of the nanoparticle is from about 45 nm to about 55 nm. In embodiments, the average longest dimension of the nanoparticle is about 51 nm.

In embodiments, the average longest dimension of the nanoparticle is from about 200 nm to about 250 nm. In embodiments, the average longest dimension of the nanoparticle is from about 400 nm to about 600 nm. In embodiments, the average longest dimension of the nanoparticle is from about 430 nm to about 530 nm.

In embodiments, the average longest dimension of the nanoparticle is from about 100 nm to about 400 nm. In embodiments, the average longest dimension of the nanoparticle is about 170 nm to 270 nm. In embodiments, the average longest dimension of the nanoparticle is about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm, 580 nm, 585 nm, 590 nm, 595 nm, or 600 nm. In embodiments, the average shortest dimension of the nanoparticle is about 10 nm.

In embodiments, the average longest dimension of the nanoparticle is from about 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm, 685 nm, 690 nm, 695 nm, 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875 nm, 880 nm, 885 nm, 890 nm, 895 nm, 900 nm, 905 nm, 910 nm, 915 nm, 920 nm, 925 nm, 930 nm, 935 nm, 940 nm, 945 nm, 950 nm, 955 nm, 960 nm, 965 nm, 970 nm, 975 nm, 980 nm, 985 nm, 990 nm, 995 nm or about 1000 nm.

In embodiments, the average longest dimension of the nanoparticle is less than about 1000 nm. In embodiments, the average longest dimension of the nanoparticle is less than about 900 nm. In embodiments, the average longest dimension of the nanoparticle is less than about 800 nm. In embodiments, the average longest dimension of the nanoparticle is less than about 700 nm. In embodiments, the average longest dimension of the nanoparticle is less than about 600 nm. In embodiments, the average longest dimension of the nanoparticle is less than about 500 nm. In embodiments, the average longest dimension of the nanoparticle is less than about 400 nm. In embodiments, the average longest dimension of the nanoparticle is less than about 300 nm. In embodiments, the average longest dimension of the nanoparticle is less than about 200 nm. In embodiments, the average longest dimension of the nanoparticle is less than about 100 nm. In embodiments, the average longest dimension of the nanoparticle is less than about 90 nm. In embodiments, the average longest dimension of the nanoparticle is less than about 80 nm. In embodiments, the average longest dimension of the nanoparticle is less than about 70 nm. In embodiments, the average longest dimension of the nanoparticle is less than about 60 nm. In embodiments, the average longest dimension of the nanoparticle is less than about 50 nm. In embodiments, the average longest dimension of the nanoparticle is less than about 40 nm. In embodiments, the average longest dimension of the nanoparticle is less than about 30 nm. In embodiments, the average longest dimension of the nanoparticle is less than about 20 nm. In embodiments, the average longest dimension of the nanoparticle is less than about 10 nm.

In embodiments, the average longest dimension of the nanoparticle is less than about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm, 580 nm, 585 nm, 590 nm, 595 nm, or 600 nm. In embodiments, the average shortest dimension of the nanoparticle is about 10 nm.

In embodiments, the average longest dimension of the nanoparticle is less than about 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm, 685 nm, 690 nm, 695 nm, 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875 nm, 880 nm, 885 nm, 890 nm, 895 nm, 900 nm, 905 nm, 910 nm, 915 nm, 920 nm, 925 nm, 930 nm, 935 nm, 940 nm, 945 nm, 950 nm, 955 nm, 960 nm, 965 nm, 970 nm, 975 nm, 980 nm, 985 nm, 990 nm, 995 nm or about 1000 nm.

In embodiments, the average longest dimension of the nanoparticle is less than 1000 nm. In embodiments, the average longest dimension of the nanoparticle is less than 900 nm. In embodiments, the average longest dimension of the nanoparticle is less than 800 nm. In embodiments, the average longest dimension of the nanoparticle is less than 700 nm. In embodiments, the average longest dimension of the nanoparticle is less than 600 nm. In embodiments, the average longest dimension of the nanoparticle is less than 500 nm. In embodiments, the average longest dimension of the nanoparticle is less than 400 nm. In embodiments, the average longest dimension of the nanoparticle is less than 300 nm. In embodiments, the average longest dimension of the nanoparticle is less than 200 nm. In embodiments, the average longest dimension of the nanoparticle is less than 100 nm. In embodiments, the average longest dimension of the nanoparticle is less than 90 nm. In embodiments, the average longest dimension of the nanoparticle is less than 80 nm. In embodiments, the average longest dimension of the nanoparticle is less than 70 nm. In embodiments, the average longest dimension of the nanoparticle is less than 60 nm. In embodiments, the average longest dimension of the nanoparticle is less than 50 nm. In embodiments, the average longest dimension of the nanoparticle is less than 40 nm. In embodiments, the average longest dimension of the nanoparticle is less than 30 nm. In embodiments, the average longest dimension of the nanoparticle is less than 20 nm. In embodiments, the average longest dimension of the nanoparticle is less than 10 nm.

In embodiments, the average longest dimension of the nanoparticle is less than 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm, 580 nm, 585 nm, 590 nm, 595 nm, or 600 nm. In embodiments, the average shortest dimension of the nanoparticle is about 10 nm.

In embodiments, the average longest dimension of the nanoparticle is less than 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm, 685 nm, 690 nm, 695 nm, 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875 nm, 880 nm, 885 nm, 890 nm, 895 nm, 900 nm, 905 nm, 910 nm, 915 nm, 920 nm, 925 nm, 930 nm, 935 nm, 940 nm, 945 nm, 950 nm, 955 nm, 960 nm, 965 nm, 970 nm, 975 nm, 980 nm, 985 nm, 990 nm, 995 nm or about 1000 nm.

In embodiments, the nanoparticle is covalently attached to one or more nanoparticle substituents, wherein the nanoparticle substituents are: (i) -L²-X¹—R³; (ii) -L²-X¹-L¹-X³; X³; or (iii) -L²-X³. X¹ is a bioconjugate linker or a bond. X³ is a bioconjugate reactive group. L¹ is a polymeric linker. L² is independently a bond, —NR^{1a}, —O—, —S—, —C(O)—, —C(O)O—, —S(O)—, —S(O)₂—, —NR^{1a}C(O)—, —C(O)NR^{1b}, —C(O)(CH₂)_{z1}—, —NR^{1a}C(O)—, —NR^{1a}C(O)NR^{1b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R^{1a} and R^{1b} are independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol z1 is an integer from 1 to 10. R³ is a polymeric moiety. In embodiments, the nanoparticle substituents does not include poly(lactate)-poly(ethylene glycol) copolymer, poly((3-amino ester), poly(lactate), poly(ethylene glycol)-dimethacrylate, or methyl ether poly(ethylene glycol)-poly((3-amino ester) copolymer. In embodiments, X¹ or X³ independently do not include biotin. In embodiments, X¹ or X² independently do not include biotin. In embodiments, -L²-X¹—R³, -L²-X¹-L¹-X³, or -L²-X³ does not include biotin. In embodiments, -L²-X¹—R³, -L²-X¹-L¹-X³, and -L²-X³ does not include biotin. In embodiments, the silica nanoparticle does not include biotin.

In embodiments, L¹ is independently a linear polymeric linker. In embodiments, L¹ is independently a branched polymeric linker. In embodiments, a nanoparticle includes multiple, optionally different, L¹ linkers and each L¹ linker is independently a linear or branched polymeric linker. In embodiments, L¹ is independently branched with 3 to 10 branches. In embodiments, L¹ is independently a divalent polyethylene glycol. In embodiments, L¹ is independently divalent PEG₄₀₀-SH. In embodiments, L¹ is independently divalent PEG₁₀₀₀-SH. In embodiments, L¹ is independently divalent PEG₂₀₀₀-SH. In embodiments, L¹ is independently divalent PEG₅₀₀₀-SH. It will be understood that the immediately preceding divalent PEG-SH groups may be bonded through the terminal thiol group where the bond between sulfur and hydrogen is replaced with a bond between sulfur and another moiety. In embodiments, L¹ is independently divalent TFP-(PEG₁₁)₃. It will be understood that the immediately preceding divalent TFP-PEG groups may be bonded through the tetrafluorophenyl (TFP) ester group where the bond is between the tetrafluorophenyl ester and another moiety. In embodiments, L¹ is independently divalent NHS-(PEG₂₄)₃. It will be understood that the immediately preceding divalent NSH-PEG groups may be bonded through the N-hydroxysuccinimide group where the bond is between N-hydroxysuccinimide and another moiety. In embodiments, L¹ is independently polyethylene glycol with an average molecular weight of about 400 g/mol, 484 g/mol, 1000 g/mol, 1450 g/mol, 1500 g/mol, 2000 g/mol, or 5000 g/mol. In embodiments, L¹ is independently polyethylene glycol with an average molecular weight of about 400 g/mol. In embodiments, L¹ is independently polyethylene glycol with an average molecular weight of about 484 g/mol. In embodiments, L¹ is independently polyethylene glycol with an average molecular weight of about 484 g/mol per arm. In embodiments, L¹ is independently polyethylene glycol with an average molecular weight of about 1000 g/mol. In embodiments, L¹ is independently polyethylene glycol with an average molecular weight of about 1450 g/mol. In embodiments, L¹ is independently polyethylene glycol with an average molecular weight of about 1500 g/mol. In embodiments, L¹ is independently polyethylene glycol with an average molecular weight of about 2000 g/mol. In embodiments, L¹ is independently polyethylene glycol with an average molecular weight of about 5000 g/mol. In embodiments, L¹ is polyethylene glycol with an average molecular weight of about 400 g/mol, 484 g/mol, 1000 g/mol, 1450 g/mol, 1500 g/mol, 2000 g/mol, or 5000 g/mol within +/−10, 20, 30, 40, or 50 of the average molecular weight.

In embodiments, L¹ is independently a polymeric linker further including a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L² is independently a bond, —NR^{1a}—, —S—, —C(O)—, —C(O)O—, —S(O)—, —S(O)₂—, —NR^{1a}C(O)—, —C(O)NR^{1b}—, —C(O)(CH₂)_{z1}—, —NR^{1a}C(O)NR^{1a}C(O)NR^{1b}—, substituted or unsubstituted alkylene (e.g. C₁-C₈ alkylene, C₁-C₆ alkylene, or C₁-C₄ alkylene), substituted or unsubstituted heteroalkylene (e.g. 2 to 10 membered heteroalkylene, 2 to 8 membered heteroalkylene, 4 to 8 membered heteroalkylene, 2 to 6 membered heteroalkylene, or 2 to 4 membered heteroalkylene), substituted or unsubstituted cycloalkylene (e.g. C₃-C₈ cycloalkylene, C₄-C₈ cycloalkylene, or C₅-C₆ cycloalkylene), substituted or unsubstituted heterocycloalkylene (e.g. 3 to 8 membered heterocycloalkylene, 4 to 8 membered heterocycloalkylene, or 5 to 6 membered heterocycloalkylene), substituted or unsubstituted arylene (e.g. C₆-C₁₀ arylene or C₆ arylene), or substituted or unsubstituted heteroarylene (e.g. 5 to 10 membered heteroarylene, 5 to 9 membered heteroarylene, or 5 to 6 membered heteroarylene). In embodiments, L² is a bond.

In embodiments, L² has the formula -L^{2A}-L^{2B}-. L^{2A} and L^{2B} are independently a bond, —NR^{1a}—, —O—, —S—, —C(O)—, —C(O)O—, —S(O)—, —S(O)₂—, —NR^{1a}C(O)—, —C(O)NR^{1b}—, —C(O)(CH₂)_{z1}—, —NR^{1a}C(O)O—, —NR^{1a}C(O)NR^{1b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{2A} is a substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L^{2B} is a substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L^{2A} and L^{2B} are independently an unsubstituted alkylene, unsubstituted heteroalkylene, unsubstituted cycloalkylene, unsubstituted heterocycloalkylene, unsubstituted arylene, or unsubstituted heteroarylene.

In embodiments, L² has the formula:

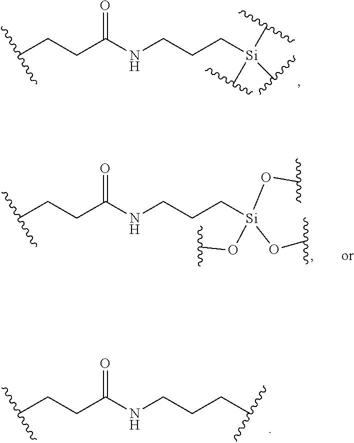 [SMILES: *CCC(=O)NCCC[Si](*)(*)* ; *CCC(=O)NCCC[Si](O*)(O*)O* ; *CCCNC(=O)CC*]

In embodiments, L² has the formula:

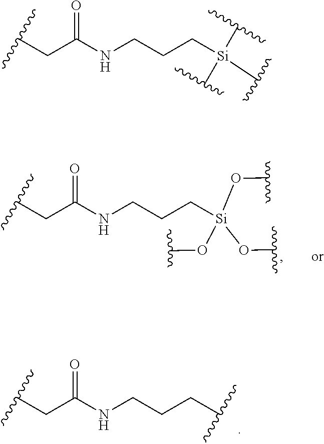 [SMILES: *CC(=O)NCCC[Si](*)(*)* ; *CC(=O)NCCC[Si](O*)(O*)O* ; *CCCNC(=O)C*]

In embodiments, L² has the formula:

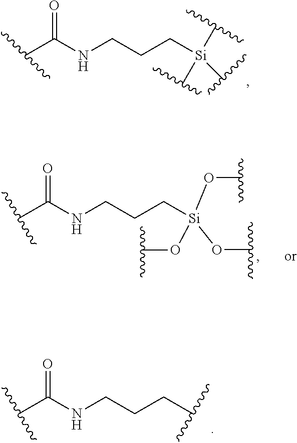 [SMILES: *C(=O)NCCC[Si](*)(*)* ; *O[Si](CCCNC(*)=O)(O*)O* ; *CCCNC(*)=O]

In embodiments, L² has the formula:

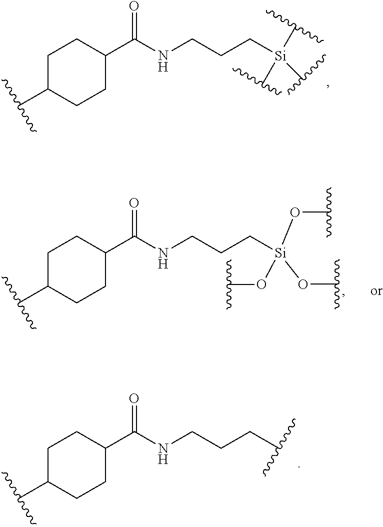 [SMILES: *C1CCC(C(=O)NCCC[Si](*)(*)*)CC1 ; *O[Si](CCCNC(=O)C1CCC(*)CC1)(O*)O* ; *CCCNC(=O)C1CCC(*)CC1]

In embodiments, L² has the formula:

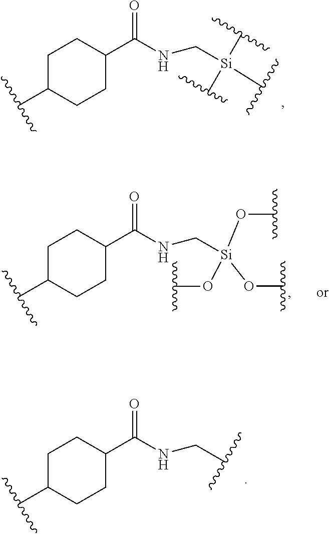 [SMILES: *C1CCC(C(=O)NC[Si](*)(*)*)CC1 ; *O[Si](CNC(=O)C1CCC(*)CC1)(O*)O* ; *CNC(=O)C1CCC(*)CC1]

In embodiments, L² has the formula:

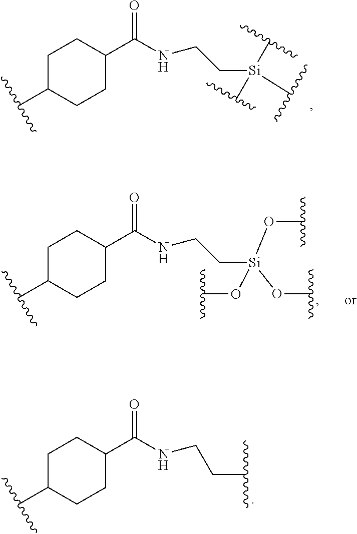 [SMILES: *C1CCC(C(=O)NCC[Si](*)(*)*)CC1 ; *O[Si](CCNC(=O)C1CCC(*)CC1)(O*)O* ; *CCNC(=O)C1CCC(*)CC1]

In embodiments, L² has the formula:

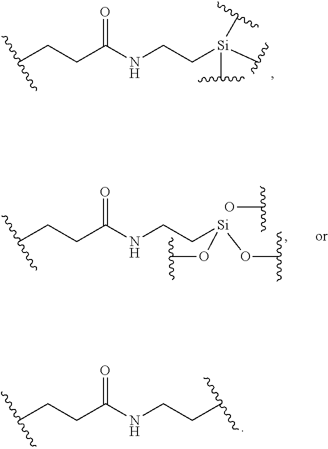

In embodiments, L² is independently a bond, —NR^{1a}—, —O—, —S—, —C(O)—, —C(O)O—, —S(O)—, —S(O)₂—, —NR^{1a}C(O)—, —C(O)NR^{1b}—, —C(O)(CH₂)_{z1}—, —NR^{1a}C(O)O—, —NR^{1a}C(O)NR^{1b}—, R⁴-substituted or unsubstituted alkylene (e.g. C₁-C₈ alkylene, C₁-C₆ alkylene, or C₁-C₄ alkylene), R⁴-substituted or unsubstituted heteroalkylene (e.g. 2 to 10 membered heteroalkylene, 2 to 8 membered heteroalkylene, 4 to 8 membered heteroalkylene, 2 to 6 membered heteroalkylene, or 2 to 4 membered heteroalkylene), R⁴-substituted or unsubstituted cycloalkylene (e.g. C₃-C₈ cycloalkylene, C₄-C₈ cycloalkylene, or C₅-C₆ cycloalkylene), R⁴-substituted or unsubstituted heterocycloalkylene (e.g. 3 to 8 membered heterocycloalkylene, 4 to 8 membered heterocycloalkylene, or 5 to 6 membered heterocycloalkylene), R⁴-substituted or unsubstituted arylene (e.g. C₆-C₁₀ arylene or C₆ arylene), or R⁴-substituted or unsubstituted heteroarylene (e.g. 5 to 10 membered heteroarylene, 5 to 9 membered heteroarylene, or 5 to 6 membered heteroarylene). In embodiments, L² is independently a bond, —NR^{1a}—, —O—, —S—, —C(O)—, —C(O)O—, —S(O)—, —S(O)₂—, —NR^{1a}C(O)—, —C(O)NR^{1b}—, —C(O)(CH₂)_{z1}—, —NR^{1a}C(O)O—, —NR^{1a}C(O)NR^{1b}—, unsubstituted alkylene (e.g. C₁-C₈ alkylene, C₁-C₆ alkylene, or C₁-C₄ alkylene), unsubstituted heteroalkylene (e.g. 2 to 10 membered heteroalkylene, 2 to 8 membered heteroalkylene, 4 to 8 membered heteroalkylene, 2 to 6 membered heteroalkylene, or 2 to 4 membered heteroalkylene), unsubstituted cycloalkylene (e.g. C₃-C₈ cycloalkylene, C₄-C₈ cycloalkylene, or C₅-C₆ cycloalkylene), unsubstituted heterocycloalkylene (e.g. 3 to 8 membered heterocycloalkylene, 4 to 8 membered heterocycloalkylene, or 5 to 6 membered heterocycloalkylene), unsubstituted arylene (e.g. C₆-C₁₀ arylene or C₆ arylene), or unsubstituted heteroarylene (e.g. 5 to 10 membered heteroarylene, 5 to 9 membered heteroarylene, or 5 to 6 membered heteroarylene).

R⁴ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁵-substituted or unsubstituted alkyl (e.g. C₁-C₈ alkyl, C₁-C₆ alkyl, or C₁-C₄ alkyl), R⁵-substituted or unsubstituted heteroalkyl (e.g. 2 to 8 membered heteroalkyl, 2 to 6 membered heteroalkyl, or 2 to 4 membered heteroalkyl), R⁵-substituted or unsubstituted cycloalkyl (e.g. C₃-C₈ cycloalkyl, C₄-C₈ cycloalkyl, or C₅-C₆ cycloalkyl), R⁵-substituted or unsubstituted heterocycloalkyl (e.g. 3 to 8 membered heterocycloalkyl, 4 to 8 membered heterocycloalkyl, or 5 to 6 membered heterocycloalkyl), R⁵-substituted or unsubstituted aryl (e.g. C₆-C₁₀ aryl or C₆ aryl), or R⁵-substituted or unsubstituted heteroaryl (e.g. 5 to 10 membered heteroaryl, 5 to 9 membered heteroaryl, or 5 to 6 membered heteroaryl).

R⁵ is independently oxo, halogen, —F, —Cl, —Br, —I, —CF₃, —CCl₃, —CN, —OH, —NH₂, —COOH, —NO₂, —SH, —SO₃H, —SO₄H, —SO₂NH₂, —NHNH₂, —ONH₂, —NHC(O)NHNH₂, —NHC═(O) NH₂, —NHSO₂H, —NHC═(O)H, —NHC(O)—OH, —NHOH, —OCF₃, —OCHF₂, unsubstituted alkyl (e.g. C₁-C₈ alkyl, C₁-C₆ alkyl, or C₁-C₄ alkyl), unsubstituted heteroalkyl (e.g. 2 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, R^{1a} and R^{1b} are independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂ substituted or unsubstituted alkyl (e.g. C₁-C₈ alkyl, C₁-C₆ alkyl, or C₁-C₄ alkyl), substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), substituted or unsubstituted cycloalkyl (e.g. C₃-C₈ cycloalkyl, C₄-C₈ cycloalkyl, or C₅-C₆ cycloalkyl), substituted or unsubstituted heterocycloalkyl (e.g. 3 to 8 membered heterocycloalkyl, 4 to 8 membered heterocycloalkyl, or 5 to 6 membered heterocycloalkyl), substituted or unsubstituted aryl (e.g. C₆-C₁₀ aryl or C₆ aryl), or substituted or unsubstituted heteroaryl (e.g. 5 to 10 membered heteroaryl, 5 to 9 membered heteroaryl, or 5 to 6 membered heteroaryl). In embodiments, R^{1a} and R^{1b} are independently hydrogen,
halogen, —CF₃, —CN, —OH, —COOH, —CONH₂, substituted or unsubstituted alkyl (e.g. C₁-C₈ alkyl, C₁-C₆ alkyl, or C₁-C₄ alkyl), substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), substituted or unsubstituted cycloalkyl (e.g. C₃-C₈ cycloalkyl, C₄-C₈ cycloalkyl, or C₅-C₆ cycloalkyl), substituted or unsubstituted heterocycloalkyl (e.g. 3 to 8 membered heterocycloalkyl, 4 to 8 membered heterocycloalkyl, or 5 to 6 membered heterocycloalkyl), substituted or unsubstituted aryl (e.g. C₆-C₁₀ aryl or C₆ aryl), or substituted or unsubstituted heteroaryl (e.g. 5 to 10 membered heteroaryl, 5 to 9 membered heteroaryl, or 5 to 6 membered heteroaryl).

In embodiments, R^{1a} is independently hydrogen,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, R⁸-substituted or unsubstituted alkyl (e.g. C₁-C₈ alkyl, C₁-C₆ alkyl, or C₁-C₄ alkyl), R⁸-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁸-substituted or unsubstituted cycloalkyl (e.g. C₃-C₈ cycloalkyl, C₄-C₈ cycloalkyl, or C₅-C₆ cycloalkyl), R⁸-substituted or unsubstituted heterocycloalkyl (e.g. 3 to 8 membered heterocycloalkyl, 4 to 8 membered heterocycloalkyl, or 5 to 6 membered heterocycloalkyl), R⁸-substituted or unsubstituted aryl (e.g. C₆-C₁₀ aryl or C₆ aryl), or R⁸-substituted or unsubstituted heteroaryl (e.g. 5 to 10 membered heteroaryl, 5 to 9 membered heteroaryl, or 5 to 6 membered heteroaryl). In embodiments, R^{1a} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, unsubstituted alkyl (e.g. C₁-C₈ alkyl, C₁-C₆ alkyl, or C C₄ alkyl), unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

R⁸ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, R⁹-substituted or unsubstituted alkyl (e.g. C₁-C₈ alkyl, C₁-C₆ alkyl, or C₁-C₄ alkyl), R⁹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁹-substituted or unsubstituted cycloalkyl (e.g. C₃-C₈ cycloalkyl, C₄-C₈ cycloalkyl, or C₅-C₆ cycloalkyl), R⁹-substituted or unsubstituted heterocycloalkyl (e.g. 3 to 8 membered heterocycloalkyl, 4 to 8 membered heterocycloalkyl, or 5 to 6 membered heterocycloalkyl), R⁹-substituted or unsubstituted aryl (e.g. C₆-C₁₀ aryl or C₆ aryl), or R⁹-substituted or unsubstituted heteroaryl (e.g. 5 to 10 membered heteroaryl, 5 to 9 membered heteroaryl, or 5 to 6 membered heteroaryl).

In embodiments, R^{1b} is independently hydrogen,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, R¹⁰-substituted or unsubstituted alkyl (e.g. C₁-C₈ alkyl, C₁-C₆ alkyl, or C₁-C₄ alkyl), R¹⁰-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R¹⁰-substituted or unsubstituted cycloalkyl (e.g. C₃-C₈ cycloalkyl, C₄-C₈ cycloalkyl, or C₅-C₆ cycloalkyl), R¹⁰-substituted or unsubstituted heterocycloalkyl (e.g. 3 to 8 membered heterocycloalkyl, 4 to 8 membered heterocycloalkyl, or 5 to 6 membered heterocycloalkyl), R¹⁰-substituted or unsubstituted aryl (e.g. C₆-C₁₀ aryl or C₆ aryl), or R¹⁰-substituted or unsubstituted heteroaryl (e.g. 5 to 10 membered heteroaryl, 5 to 9 membered heteroaryl, or 5 to 6 membered heteroaryl). In embodiments, R^{1b} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, unsubstituted alkyl (e.g. C₁-C₈ alkyl, C₁-C₆ alkyl, or C₁-C₄ alkyl), unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

R¹⁰ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, R¹¹-substituted or unsubstituted alkyl (e.g. C₁-C₈ alkyl, C₁-C₆ alkyl, or C₁-C₄ alkyl), R¹¹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R¹¹-substituted or unsubstituted cycloalkyl (e.g. C₃-C₈ cycloalkyl, C₄-C₈ cycloalkyl, or C₅-C₆ cycloalkyl), R¹¹-substituted or unsubstituted heterocycloalkyl (e.g. 3 to 8 membered heterocycloalkyl, 4 to 8 membered heterocycloalkyl, or 5 to 6 membered heterocycloalkyl), R¹¹-substituted or unsubstituted aryl (e.g. C₆-C₁₀ aryl or C₆ aryl), or R¹¹-substituted or unsubstituted heteroaryl (e.g. 5 to 10 membered heteroaryl, 5 to 9 membered heteroaryl, or 5 to 6 membered heteroaryl).

In embodiments, R⁹ and R¹¹ are independently oxo,

halogen, —F, —Cl, —Br, —I, —CF₃, —CCl₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC═(O) NH₂, —NHSO₂H, —NHC═(O)H, —NHC(O)—OH, —NHOH, —OCF₃, —OCHF₂, unsubstituted alkyl (e.g. C₁-C₈ alkyl, C₁-C₆ alkyl, or C₁-C₄ alkyl), unsubstituted heteroalkyl (e.g. 2 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, R³ is monovalent polyethylene glycol (PEG). In embodiments, when there are more than one R³ groups present, each R³ group is optionally different. In embodiments, R³ is monovalent PEG₄₀₀-SH. In embodiments, R³ is monovalent PEG₁₀₀₀-SH. In embodiments, R³ is monovalent PEG₂₀₀₀-SH. In embodiments, R³ is monovalent PEG₅₀₀₀-SH. It will be understood that the immediately preceding monovalent PEG-SH groups may be bonded through the terminal thiol group where the bond between sulfur and hydrogen is replaced with a bond between sulfur and another moiety. In embodiments, R³ is monovalent TFP-(PEG₁₁)₃. It will be understood that the immediately preceding monovalent TFP-PEG groups may be bonded through the tetrafluorophenyl (TFP) ester group where the bond is between the tetrafluorophenyl ester and another moiety. In embodiments, R³ is monovalent NSH-(PEG₂₄)₃. It will be understood that the immediately preceding monovalent NSH-PEG groups may be bonded through the N-hydroxysuccinimide group where the bond is between N-hydroxysuccinimide and another moiety. In embodiments, R³ is a monovalent polyethylene glycol with an average molecular weight of about 400 g/mol, 484 g/mol, 1000 g/mol, 1450 g/mol, 1500 g/mol, 2000 g/mol, or 5000 g/mol. In embodiments, R³ is a monovalent polyethylene glycol with an average molecular weight of about 400 g/mol. In embodiments, R³ is a monovalent polyethylene glycol with an average molecular weight of about 484 g/mol. In embodiments, R³ is a monovalent polyethylene glycol with an average molecular weight of about 484 g/mol per arm. In embodiments, R³ is a monovalent polyethylene glycol with an average molecular weight of about 1000 g/mol. In embodiments, R³ is a monovalent polyethylene glycol with an average molecular weight of about 1450 g/mol. In embodiments, R³ is a monovalent polyethylene glycol with an average molecular weight of about 1500 g/mol. In embodiments, R³ is a monovalent polyethylene glycol with an average molecular weight of about 2000 g/mol. In embodiments, R³ is a monovalent polyethylene glycol with an average molecular weight of about 5000 g/mol.

In embodiments, z1 is independently 10. In embodiments, z1 is independently 9. In embodiments, z1 is independently 8. In embodiments, z1 is independently 7. In embodiments, z1 is independently 6. In embodiments, z1 is independently 5. In embodiments, z1 is independently 4. In embodiments, z1 is independently 3. In embodiments, z1 is independently 2. In embodiments, z1 is independently 1.

In embodiments, the detectable agent is a radioisotope, fluorophore, electron-dense reagent, enzyme, biotin, paramagnetic agent, or magnetic agent. In embodiments, the detectable agent is a fluorophore. In embodiments, the detectable agent includes a cyanine, heptamethine, xanthene, rhodamine, fluorescein, boron-dipyrromethene, boron dipyridyl, naphthalene, coumarin, acridine, acridinium, tetrapyrrole, tetraphenylethene, oxazine, pyrene, oxadiazole, subphthalocyanine, carbopyrinin, benzopyrinium, or phthalocyanine. In embodiments, the detectable agent is within the nanoparticle. In embodiments, the detectable agent is on the surface of the nanoparticle. In embodiments, the detectable agent is attached to the surface of the nanoparticle. In embodiments, the detectable agent is not on the surface of the nanoparticle.

In embodiments, the detectable agent is a fluorophore having a maximum emission wavelength from about 495 nm to about 570 nm. In embodiments, the detectable agent is a fluorophore having a maximum emission wavelength from about 570 nm to about 620 nm. In embodiments, the detectable agent is a fluorophore having a maximum emission wavelength from about 620 nm to about 650 nm. In embodiments, the detectable agent is a fluorophore having a maximum emission wavelength from about 710 nm to about 850 nm. In embodiments, the detectable agent is a fluorophore having a maximum emission wavelength from about 850 nm to about 1350 nm. In embodiments, the detectable agent is indocyanine green. In embodiments, the fluorophore is an FDA approved dye for clinical use, or has a low toxicity profile. One skilled in the art would recognize that common fluorescent proteins or non-protein organic fluorophores may be used. In embodiments, the detectable agent is a fluorophore having a maximum emission wavelength from about 495 nm to about 595 nm. In embodiments, the detectable agent is a fluorophore having a maximum emission wavelength from about 495 nm to about 585 nm. In embodiments, the detectable agent is a fluorophore having a maximum emission wavelength from about 510 nm to about 585 nm. In embodiments, the detectable agent is a fluorophore having a maximum emission wavelength of 510 nm. In embodiments, the detectable agent is a fluorophore having a maximum emission wavelength of 585 nm.

In embodiments, the detectable agent is a fluorophore having an emission wavelength from about 495 nm to about 570 nm. In embodiments, the detectable agent is a fluorophore having an emission wavelength from about 570 nm to about 620 nm. In embodiments, the detectable agent is a fluorophore having an emission wavelength from about 620 nm to about 650 nm. In embodiments, the detectable agent is a fluorophore having an emission wavelength from about 710 nm to about 850 nm. In embodiments, the detectable agent is a fluorophore having an emission wavelength from about 850 nm to about 1350 nm. In embodiments, the detectable agent is indocyanine green. In embodiments, the fluorophore is an FDA approved dye for clinical use, or has a low toxicity profile. In embodiments, the detectable agent is a fluorophore having an emission wavelength from about 495 nm to about 595 nm. In embodiments, the detectable agent is a fluorophore having an emission wavelength from about 495 nm to about 585 nm. In embodiments, the detectable agent is a fluorophore having an emission wavelength from about 510 nm to about 585 nm. In embodiments, the detectable agent is a fluorophore having an emission wavelength of 510 nm. In embodiments, the detectable agent is a fluorophore having an emission wavelength of 585 nm.

In embodiments, the silica nanoparticle further includes a stabilizing agent. In embodiments, the stabilizing agent is conjugated directly to the silica nanoparticle. In embodiments, the stabilizing agent is conjugated to the silica nanoparticle. In embodiments, the stabilizing agent is a surfactant or a polymer. In embodiments, the stabilizing agent is a cationic polymer. In embodiments, the stabilizing agent is selected from hydrophilic sterically repulsive groups (for example, oligo(ethylene glycol), oligosaccharides, etc.), cationically charged groups (for example, amines), anionically charged groups (for example, sulfonates, carboxylic acids, phosphonates, phosphates, etc.) and zwitterionically charged groups (e.g., amino-phosphonates, amino-sulfonates, such as N,N-dimethyl-N-acryloyloxyethyl-N-(3-sulfopropyl)-ammonium betaine, N,N-dimethyl-N-acrylamidopropyl N-(3-sulfopropyl)-ammonium betaine, 2-(methylthio)ethyl methacryloyl-S-(sulfopropyl)-sulfonium betaine, 2-[(2-acryloylethyl)dimethylammonio]ethyl 2-methyl phosphate, 2-(acryloyloxyethyl)-2′-(trimethylammonium)ethyl phosphate, 2-methacryloyloxyethyl phosphorylcholine (MPC), 2-[(3-acrylamidopropyl)dimethylammonio]ethyl 2′-isopropyl phosphate (AAPI), 1-vinyl-3-(3-sulfopropyl)imidazolium hydroxide, 1-(3-sulfopropyl)-2-vinylpyridinium betaine, N-(4-sulfobutyl)-N methyl-N,N-diallylamine ammonium betaine (M DABS), N,N-diallyl-N-methyl-N-(2-sulfoethyl) ammonium betaine, N,N-dimethyl-N-(3-methacrylamidopropyl)-N-(3-sulfopropyl) ammonium betaine, N,N-dimethyl-N-acryloyloxyethyl-N-(3-sulfopropyl)-ammonium betaine, N,N-dimethyl-N acrylamidopropyl-N-(2-carboxymethyl)-ammonium betaine, N,N-dimethyl-N-methacryloyloxyethyl-N-(3-sulfopropyl)-ammonium betaine, and N, N-dimethyl-N-(3-methacrylamidopropyl)-N-(3-sulfopropyl) ammonium betaine), PBS, or combinations thereof. In embodiments, the stabilizing agent is a polymer (e.g., polyoxazoline polymer), chitosan, poly-L-lysine or polyethylenimine (PEI). In embodiments, the stabilizing agent is polysorbate 20 or polysorbate 80. In embodiments, the stabilizing agent is a salt. In embodiments, the stabilizing agent is NaCl in a PBS solution. In embodiments, the stabilizing agent is PBS. In embodiments, the stabilizing agent is NaCl.

In embodiments the detectable agent and stabilizing agent may be present in mass ratios of, for example, but not limited to, 1:2; 1:3; 1:4, 1:5. 1:6, 1:7, 1:8, 1:9, 1:10, 1:11, 1:12, 1:13, 1:14, or 1:15 (detectable agent:stabilizing agent). In embodiments the detectable agent and stabilizing agent may be present in molar ratios of, for example, but not limited to, 1:2; 1:3; 1:4, 1:5. 1:6, 1:7, 1:8, 1:9, 1:10, 1:11, 1:12, 1:13, 1:14, or 1:15 (detectable agent:stabilizing agent). The ratio of the detectable agent to the stabilizing agent depends upon many factors, for example, the overall size, the size/weight of the detectable agent, the hydrophobicity of the detectable agent, or the number of detectable agent molecules in the nanoparticle.

In embodiments, the nanoparticle is covalently attached to one or more nanoparticle substituents. In embodiments, the nanoparticle substituent includes a polymeric moiety. In embodiments, the polymeric moiety is a polyethylene glycol moiety. In embodiments, the nanoparticle substituents occupy about 5%, 10%, 15%, 20%, 25%, 40%, 50%, 60%, 75%, 80%, 90%, 95%, 99%, or about 100% of the nanoparticle surface. In embodiments, the polymeric linker does not include poly(lactate)-poly(ethylene glycol) copolymer, poly(β-amino ester), poly(lactate), poly(ethylene glycol)-dimethacrylate, or methyl ether poly(ethylene glycol)-poly(β-amino ester) copolymer.

In embodiments, the nanoparticle includes a plurality of nanoparticle substituents of the formula (i) and a plurality of nanoparticle substituents of the formula (ii), and not formula (iii). In embodiments, the nanoparticle includes a plurality of nanoparticle substituents of the formula (i) and a plurality of nanoparticle substituents of the formula (iii), and not formula (ii). In embodiments, the nanoparticle includes a plurality of nanoparticle substituents of the formula (ii) and a plurality of nanoparticle substituents of the formula (iii), and not formula (i). In embodiments, the nanoparticle includes a plurality of nanoparticle substituents of the formula (i), and not formula (ii) or formula (iii). In embodiments, the nanoparticle includes a plurality of nanoparticle substituents of the formula (ii), and not formula (i) or formula (iii). In embodiments, the nanoparticle includes a plurality of nanoparticle substituents of the formula (iii), and not formula (i) or formula (ii).

In embodiments, the nanoparticle includes a plurality of nanoparticle substituents of the formula (i) and a plurality of nanoparticle substituents of the formula (ii) in a ratio of about 50:50 to about 80:20. In embodiments, the ratio of a plurality of nanoparticle substituents of the formula (i) and a plurality of substituents of the formula (ii) is about 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, or 80:20.

In embodiments, the nanoparticle includes a plurality of nanoparticle substituents of the formula (i) and a plurality of nanoparticle substituents of the formula (iii) in a ratio of about 50:50 to about 80:20. In embodiments, the ratio of a plurality of nanoparticle substituents of the formula (i) and a plurality of substituents of the formula (iii) is about 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, or 80:20.

In embodiments, the nanoparticle includes a plurality of nanoparticle substituents of the formula (ii) and a plurality of nanoparticle substituents of the formula (iii) in a ratio of about 50:50 to about 80:20. In embodiments, the ratio of a plurality of nanoparticle substituents of the formula (ii) and a plurality of nanoparticle substituents of the formula (iii) is about 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, or 80:20.

In embodiments, the nanoparticle includes a plurality of nanoparticle substituents of the formula (i) and a plurality of nanoparticle substituents of the formula (ii) in a molar ratio of about 50:50 to about 80:20. In embodiments, the molar ratio of a plurality of nanoparticle substituents of the formula (i) and a plurality of substituents of the formula (ii) is about 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, or 80:20.

In embodiments, the nanoparticle includes a plurality of nanoparticle substituents of the formula (i) and a plurality of nanoparticle substituents of the formula (iii) in a molar ratio of about 50:50 to about 80:20. In embodiments, the molar ratio of a plurality of nanoparticle substituents of the formula (i) and a plurality of substituents of the formula (iii) is about 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, or 80:20.

In embodiments, the nanoparticle includes a plurality of nanoparticle substituents of the formula (ii) and a plurality of nanoparticle substituents of the formula (iii) in a molar ratio of about 50:50 to about 80:20. In embodiments, the molar ratio of a plurality of nanoparticle substituents of the formula (ii) and a plurality of nanoparticle substituents of the formula (iii) is about 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, or 80:20.

In an aspect is provided a cell including a nanoparticle (e.g., a silica nanoparticle) described herein. In embodiments, the cell is a tumor tropic cell, macrophage, stem cell (e.g., neural, mesenchymal), or T-cell. In embodiments, the cell is neural stem cell, a mesenchymal stem cell, a mesenchymal stromal cell, a hematopoetic stem cell, T-lymphocyte, a macrophage, or a liver stem cell. In embodiments, the cell is a neural stem cell. In embodiments, the cell is genetically modified. In embodiments, the cell is a genetically modified stem cell. In embodiments, the cell is a genetically modified neural stem cell. In embodiments, the neural stem cell is a human HB1.F3 stem cell. In embodiments, the nanoparticle is within the cell. In embodiments, the nanoparticle is incorporated within the cell via the enhanced permeability and retention (EPR) effect. In embodiments, the nanoparticle is an unmodified silica nanoparticle and the cell is a neural stem cell.

In an aspect is provided a nanoparticle-cell construct including a nanoparticle covalently attached to a protein (e.g., a cell-surface protein) through a covalent linker. In embodiments, the protein is attached to cell and is a cell surface protein. In embodiments, the protein includes a sulfur-containing amino acid. In embodiments, the protein includes methionine, cysteine, homocysteine, or taurine. In embodiments, the protein includes a sulfhydryl moiety. In embodiments of the nanoparticle-cell construct, the nanoparticle is a silica nanoparticle. In embodiments of the nanoparticle-cell construct, the nanoparticle is a silica nanoparticle which comprises a detectable agent.

In embodiments, the covalent linker has the formula: -L²-X¹-L¹-X²-L³ (Ia) or -L²-X²-L³(Ib). X¹ and X² are independently a bioconjugate linker or a bond, wherein at least one of X¹ or X² is a bioconjugate linker; L¹ is independently a polymeric linker; L² is independently a bond, —NR^{1a}—, —O—, —S—, —C(O)—, —C(O)O—, —S(O)—, —S(O)₂—, —NR^{1a}C(O)—, —C(O)NR^{1b}—, —C(O)(CH₂)_{z1}—, —NR^{1a}C(O)O—, —NR^{1a}C(O)NR^{1b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L³ is independently a bond, —NR^{2a}—, —O—, —S—, —C(O)—, —C(O)O—, —S(O)—, —S(O)₂—, —NR^{2a}C(O)—, —C(O)NR^{2b}—, —C(O)(CH₂)_{z2}—, —NR^{2a}C(O)O—, —NR^{2a}C(O)NR^{2b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R^{1a}, R^{2a}, R^{1b}, and R^{2b} are independently hydrogen,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; the symbols z1 and z2 are independently an integer from 1 to 10. In embodiments of the nanoparticle-cell construct, the polymeric linker does not include poly(lactate)-poly(ethylene glycol) copolymer, poly((3-amino ester), poly(lactate), poly(ethylene glycol)-dimethacrylate, or methyl ether poly(ethylene glycol)-poly((3-amino ester) copolymer. In embodiments, X¹ or X² independently do not include biotin. In embodiments, X¹ or X² independently do not include biotin. In embodiments, -L²-X¹-L¹-X²-L³- and -L²-X²-L³- does not include biotin. In embodiments, the silica nanoparticle does not include biotin.

In embodiments, L³ is independently a bond, —NR^{2a}—, —O—, —S—, —C(O)—, —C(O)O—, —S(O)—, —S(O)₂—, —NR^{2a}C(O)—, —C(O)NR^{2b}—, —C(O)(CH₂)_{z2}—, —NR^{2a}C(O)O—, —NR^{2a}C(O)NR^{2b}—, substituted or unsubstituted alkylene (e.g. C₁-C₈ alkylene, C₁-C₆ alkylene, or C₁-C₄ alkylene), substituted or unsubstituted heteroalkylene (e.g. 2 to 10 membered heteroalkylene, 2 to 8 membered heteroalkylene, 4 to 8 membered heteroalkylene, 2 to 6 membered heteroalkylene, or 2 to 4 membered heteroalkylene), substituted or unsubstituted cycloalkylene (e.g. C₃-C₈ cycloalkylene, C₄-C₈ cycloalkylene, or C₅-C₆ cycloalkylene), substituted or unsubstituted heterocycloalkylene (e.g. 3 to 8 membered heterocycloalkylene, 4 to 8 membered heterocycloalkylene, or 5 to 6 membered heterocycloalkylene), substituted or unsubstituted arylene (e.g. C₆-C₁₀ arylene or C₆ arylene), or substituted or unsubstituted heteroarylene (e.g. 5 to 10 membered heteroarylene, 5 to 9 membered heteroarylene, or 5 to 6 membered heteroarylene). In embodiments, L³ is a bond.

In embodiments, L³ is independently a bond, —NR^{2a}—, —O—, —S—, —C(O)—, —C(O)O—, —S(O)—, —S(O)₂—, —NR^{2a}C(O)—, —C(O)NR^{2b}—, —C(O)(CH₂)_{z2}—, —NR^{2a}C(O)O—, —NR^{2a}C(O)NR^{2b}—, R⁶-substituted or unsubstituted alkylene (e.g. C₁-C₈ alkylene, C₁-C₆ alkylene, or C₁-C₄ alkylene), R⁶-substituted or unsubstituted heteroalkylene (e.g. 2 to 10 membered heteroalkylene, 2 to 8 membered heteroalkylene, 4 to 8 membered heteroalkylene, 2 to 6 membered heteroalkylene, or 2 to 4 membered heteroalkylene), R⁶-substituted or unsubstituted cycloalkylene (e.g. C₃-C₈ cycloalkylene, C₄-C₈ cycloalkylene, or C₅-C₆ cycloalkylene), R⁶-substituted or unsubstituted heterocycloalkylene (e.g. 3 to 8 membered heterocycloalkylene, 4 to 8 membered heterocycloalkylene, or 5 to 6 membered heterocycloalkylene), R⁶-substituted or unsubstituted arylene (e.g. C₆-C₁₀ arylene or C₆ arylene), or R⁶-substituted or unsubstituted heteroarylene (e.g. 5 to 10 membered heteroarylene, 5 to 9 membered heteroarylene, or 5 to 6 membered heteroarylene). In embodiments, L³ is independently a bond, —NR^{2a}—, —O—, —S—, —C(O)—, —C(O)O—, —S(O)—, —S(O)₂—, —NR^{2a}C(O)—, —C(O) NR^{2b}—, —C(O)(CH₂)_{z2}—, —NR^{2a}C(O)O—, —NR^{2a}C(O)NR^{2b}—, R⁶-substituted or unsubstituted alkylene (e.g. C₁-C₈ alkylene, C₁-C₆ alkylene, or C₁-C₄ alkylene), unsubstituted heteroalkylene (e.g. 2 to 10 membered heteroalkylene, 2 to 8 membered heteroalkylene, 4 to 8 membered heteroalkylene, 2 to 6 membered heteroalkylene, or 2 to 4 membered heteroalkylene), unsubstituted cycloalkylene (e.g. C₃-C₈ cycloalkylene, C₄-C₈ cycloalkylene, or C₅-C₆ cycloalkylene), unsubstituted heterocycloalkylene (e.g. 3 to 8 membered heterocycloalkylene, 4 to 8 membered heterocycloalkylene, or 5 to 6 membered heterocycloalkylene), unsubstituted arylene (e.g. C₆-C₁₀ arylene or C₆ arylene), or unsubstituted heteroarylene (e.g. 5 to 10 membered heteroarylene, 5 to 9 membered heteroarylene, or 5 to 6 membered heteroarylene).

R⁶ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, le-substituted or unsubstituted alkyl (e.g. C₁-C₈ alkyl, C₁-C₆ alkyl, or C₁-C₄ alkyl), R⁷-substituted or unsubstituted heteroalkyl (e.g. 2 to 8 membered heteroalkyl, 2 to 6 membered heteroalkyl, or 2 to 4 membered heteroalkyl), R⁷-substituted or unsubstituted cycloalkyl (e.g. C₃-C₈ cycloalkyl, C₄-C₈ cycloalkyl, or C₅-C₆ cycloalkyl), R⁷-substituted or unsubstituted heterocycloalkyl (e.g. 3 to 8 membered heterocycloalkyl, 4 to 8 membered heterocycloalkyl, or 5 to 6 membered heterocycloalkyl), R⁷-substituted or unsubstituted aryl (e.g. C₆-C₁₀ aryl or C₆ aryl), or R⁷-substituted or unsubstituted heteroaryl (e.g. 5 to 10 membered heteroaryl, 5 to 9 membered heteroaryl, or 5 to 6 membered heteroaryl).

R⁷ is independently oxo,

halogen, —F, —Cl, —Br, —I, —CF₃, —CCl₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl (e.g. C₁-C₈ alkyl, C₁-C₆ alkyl, or C₁-C₄ alkyl), unsubstituted heteroalkyl (e.g. 2 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, R^{2a} and R^{2b} are independently hydrogen,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂ substituted or unsubstituted alkyl (e.g. C₁-C₈ alkyl, C₁-C₆ alkyl, or C₁-C₄ alkyl), substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), substituted or unsubstituted cycloalkyl (e.g. C₃-C₈ cycloalkyl, C₄-C₈ cycloalkyl, or C₅-C₆ cycloalkyl), substituted or unsubstituted heterocycloalkyl (e.g. 3 to 8 membered heterocycloalkyl, 4 to 8 membered heterocycloalkyl, or 5 to 6 membered heterocycloalkyl), substituted or unsubstituted aryl (e.g. C₆-C₁₀ aryl or C₆ aryl), or substituted or unsubstituted heteroaryl (e.g. 5 to 10 membered heteroaryl, 5 to 9 membered heteroaryl, or 5 to 6 membered heteroaryl).

In embodiments, R^{2a} is independently hydrogen,

halogen, —CF₃, —CN, —OH, —NH₂, —COOH, —CONH₂, —NO₂, —SH, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂ R¹²-substituted or unsubstituted alkyl (e.g. C₁-C₈ alkyl, C₁-C₆ alkyl, or C₁-C₄ alkyl), R¹²-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R¹²-substituted or unsubstituted cycloalkyl (e.g. C₃-C₈ cycloalkyl, C₄-C₈ cycloalkyl, or C₅-C₆ cycloalkyl), R¹²-substituted or unsubstituted heterocycloalkyl (e.g. 3 to 8 membered heterocycloalkyl, 4 to 8 membered heterocycloalkyl, or 5 to 6 membered heterocycloalkyl), R¹²-substituted or unsubstituted aryl (e.g. C₆-C₁₀ aryl or C₆ aryl), or R¹²-substituted or unsubstituted heteroaryl (e.g. 5 to 10 membered heteroaryl, 5 to 9 membered heteroaryl, or 5 to 6 membered heteroaryl).

R¹² is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, R¹³-substituted or unsubstituted alkyl (e.g. C₁-C₈ alkyl, C₁-C₆ alkyl, or C₁-C₄ alkyl), R¹³-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R¹³-substituted or unsubstituted cycloalkyl (e.g. C₃-C₈ cycloalkyl, C₄-C₈ cycloalkyl, or C₅-C₆ cycloalkyl), R¹³-substituted or unsubstituted heterocycloalkyl (e.g. 3 to 8 membered heterocycloalkyl, 4 to 8 membered heterocycloalkyl, or 5 to 6 membered heterocycloalkyl), R¹³-substituted or unsubstituted aryl (e.g. C₆-C₁₀ aryl or C₆ aryl), or R¹³-substituted or unsubstituted heteroaryl (e.g. 5 to 10 membered heteroaryl, 5 to 9 membered heteroaryl, or 5 to 6 membered heteroaryl).

In embodiments, R^{2b} is independently hydrogen,

halogen, —CF₃, —CN, —OH, —NH₂, —COOH, —CONH₂, —NO₂, —SH, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂ R¹⁴-substituted or unsubstituted alkyl (e.g. C₁-C₈ alkyl, C₁-C₆ alkyl, or C₁-C₄ alkyl), R¹⁴-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R¹⁴-substituted or unsubstituted cycloalkyl (e.g. C₃-C₈ cycloalkyl, C₄-C₈ cycloalkyl, or C₅-C₆ cycloalkyl), R¹⁴-substituted or unsubstituted heterocycloalkyl (e.g. 3 to 8 membered heterocycloalkyl, 4 to 8 membered heterocycloalkyl, or 5 to 6 membered heterocycloalkyl), R¹⁴-substituted or unsubstituted aryl (e.g. C₆-C₁₀ aryl or C₆ aryl), or R¹⁴-substituted or unsubstituted heteroaryl (e.g. 5 to 10 membered heteroaryl, 5 to 9 membered heteroaryl, or 5 to 6 membered heteroaryl).

R¹⁴ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, R¹⁵-substituted or unsubstituted alkyl (e.g. C₁-C₈ alkyl, C₁-C₆ alkyl, or C₁-C₄ alkyl), R¹⁵-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R¹⁵-substituted or unsubstituted cycloalkyl (e.g. C₃-C₈ cycloalkyl, C₄-C₈ cycloalkyl, or C₅-C₆ cycloalkyl), R¹⁵-substituted or unsubstituted heterocycloalkyl (e.g. 3 to 8 membered heterocycloalkyl, 4 to 8 membered heterocycloalkyl, or 5 to 6 membered heterocycloalkyl), R¹⁵-substituted or unsubstituted aryl (e.g. C₆-C₁₀ aryl or C₆ aryl), or R¹⁵-substituted or unsubstituted heteroaryl (e.g. 5 to 10 membered heteroaryl, 5 to 9 membered heteroaryl, or 5 to 6 membered heteroaryl).

In embodiments, R¹³ and R¹⁵ are independently oxo,

halogen, —F, —Cl, —Br, —I, —CF₃, —CCl₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl (e.g. C₁-C₈ alkyl, C₁-C₆ alkyl, or C₁-C₄ alkyl), unsubstituted heteroalkyl (e.g. 2 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, the nanoparticle substituent is: -L²-X¹—R³ (i); -L²-X¹-L¹-X³ (ii); or L²-X³ (iii). L¹, L², X¹, and X³ are as defined herein and are optionally different. R³ is independently a polymeric moiety. X³ is independently a bioconjugate reactive group. In embodiments, when there is a plurality of L¹, L², X¹ and X³, L¹, L², X¹ and X³ are the same. In embodiments, when there is a plurality of L¹, L², X¹ and X³, L¹, L², X¹ and X³ are optionally different.

In embodiments, —X² has the formula:

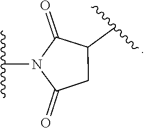 [SMILES: *C1CC(=O)N(*)C1=O]

In embodiments, —X²-L³ has the formula:

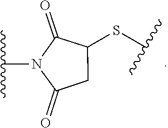 [SMILES: *SC1CC(=O)N(*)C1=O]

In embodiments, —X³ is NH₂, —COOH, —N-hydroxysuccinimide, or maleimide. In embodiments, X³ is

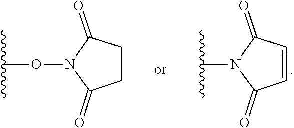 [SMILES: *ON1C(=O)CCC1=O ; *N1C(=O)C=CC1=O]

In embodiments, X³ is haloacetyl (eg., iodoacetyl, bromoacetyl, or chloroacetyl). In embodiments, X³ is pyridyl. In embodiments, X³ is -maleimide. In embodiments, X³ is —N-hydroxysuccinimide. In embodiments, X³ is —COOH. In embodiments, X³ is —NH₂.

In embodiments, z2 is independently 10. In embodiments, z2 is independently 9. In embodiments, z2 is independently 8. In embodiments, z2 is independently 7. In embodiments, z2 is independently 6. In embodiments, z2 is independently 5. In embodiments, z2 is independently 4. In embodiments, z2 is independently 3. In embodiments, z2 is independently 2. In embodiments, z2 is independently 1.

In embodiments, the nanoparticle is an inorganic nanoparticle. In embodiments, the nanoparticle is a silica nanoparticle. The nanoparticle may be an inorganic nanoparticle. The inorganic nanoparticle may be a metal nanoparticle. The metal oxide nanoparticle may be titanium oxide or zirconium oxide. In embodiments, the nanoparticle is a gold nanoparticle. In embodiments, the nanoparticle is an iron nanoparticle. In embodiments, the nanoparticle is an iron oxide nanoparticle.

In embodiments, the nanoparticle-protein construct has the formula:

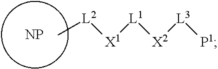

wherein NP is a nanoparticle and P¹ is a protein optionally attached to a cell (e.g., a stem cell). L², X¹, L¹, X², and L³ are as described herein. In embodiments, P¹ is attached to cell and is a cell surface protein.

In embodiments, the nanoparticle is a silica nanoparticle. The nanoparticle may be an inorganic nanoparticle. The inorganic nanoparticle may be a metal nanoparticle. The metal oxide nanoparticle may be titanium oxide or zirconium oxide. The nanoparticle may be titanium. The nanoparticle may be gold. The nanoparticle may be iron. The nanoparticle may be iron oxide.

In embodiments, the protein is a cell surface protein. In embodiments the protein is in contact with the extracellular matrix (e.g., extracellular matrix associated with a cancer cell or in contact with a cancer cell). In embodiments, the protein is in contact with a tumor. In embodiments, the nanoparticle is in contact with a tumor. In embodiments, the tumor includes stromal cells, immune cells, proteins, and extracellular matrix generated by stromal or immune cells. In embodiments, immune cells, stromal cells, proteins associate with the immune cells, proteins associated with the stromal cells, and the extracellular matrix generated from immune cells and stromal cells form part of a tumor. In embodiments, the nanoparticle is incorporated within the cell. In embodiments, the nanoparticle is incorporated within the cell via the enhanced permeability and retention (EPR) effect.

In embodiments, the linker is formed by a conjugation or bioconjugation reaction combining a first reactant moiety covalently bonded to the polymeric linker and a second reactant moiety covalently bonded to a protein. In such embodiments, the compound formed by such conjugation or bioconjugation reaction (including compositions as described herein) may be referred to as a conjugate.

In embodiments, the density of the nanoparticle is about 2.0 g/ccm. In embodiments, the density of the nanoparticle is about 1.9 g/ccm. In embodiments, the density of the nanoparticle is about 1.8 g/ccm. In embodiments, the density of the nanoparticle is about 2.1 g/ccm. In embodiments, the density of the nanoparticle is about 2.2 g/ccm.

### III. Pharmaceutical Compositions

In another aspect, is provided a pharmaceutical composition including a pharmaceutically acceptable excipient and a nanoparticle as described herein or a pharmaceutically acceptable salt thereof. In embodiments is provided a pharmaceutical composition including a pharmaceutically acceptable excipient and a silica nanoparticle as described herein or a pharmaceutically acceptable salt thereof. In embodiments is provided a pharmaceutical composition including a pharmaceutically acceptable excipient and an unmodified silica nanoparticle as described herein or a pharmaceutically acceptable salt thereof.

The compositions (e.g., nanoparticles described herein) of the present invention can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions (e.g., nanoparticles described herein) of the present invention can also be administered by injection, that is, intravenously, intramuscularly, intracutaneously, subcutaneously, intraduodenally, or intraperitoneally. Also, the compositions (e.g., nanoparticles described herein) described herein can be administered by inhalation, for example, intranasally. Additionally, the compositions (e.g., nanoparticles described herein) of the present invention can be administered transdermally. It is also envisioned that multiple routes of administration (e.g., intramuscular, oral, transdermal) can be used to administer the compositions (e.g., nanoparticles described herein) of the invention. Accordingly, the present invention also provides pharmaceutical compositions including a pharmaceutically acceptable excipient and one or more compositions (e.g., nanoparticles described herein) of the invention.

Pharmaceutical compositions provided by the present invention include compositions wherein the active ingredient (e.g., nanoparticles described herein) is contained in a therapeutically effective amount, i.e., in an amount effective to achieve its intended purpose. The actual amount effective for a particular application will depend, inter alia, on the condition being observed. When administered in methods to detect a cell, such compositions will contain an amount of active ingredient effective to achieve the desired result, e.g., detecting a cancer cell (e.g., ovarian cancer cell). Determination of a diagnostically effective amount of a compositions (e.g., nanoparticles described herein) of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., cancer, ovarian cancer, bladder cancer, head and neck cancer, brain cancer, breast cancer, lung cancer, cervical cancer, bone cancer, spinal cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, or prostate cancer), kind of concurrent treatment, complications from the disease being treated or other health-related problems. Other therapeutic regimens or agents can be used in conjunction with the methods and compositions (e.g., nanoparticles described herein) described herein. Adjustment and manipulation of established dosages (e.g., frequency and duration) are well within the ability of those skilled in the art.

The exact formulation, route of administration and dosage can be chosen by the individual physician in view of the patient's condition and the particular method in which the compositions (e.g., nanoparticles described herein) is used.

The neutral forms of the compositions (e.g., nanoparticles described herein) may be regenerated by contacting the salt with a base or acid and isolating the parent compositions (e.g., nanoparticles described herein) in the conventional manner. The parent form of the compositions (e.g., nanoparticles described herein) may differ from the various salt forms in certain physical properties, such as solubility in polar solvents, but otherwise the salts are equivalent to the parent form of the compositions (e.g., nanoparticles described herein) for the purposes of the present invention.

Certain compositions described herein of the present invention can exist in unsolvated forms as well as solvated forms, including hydrated forms. In general, the solvated forms are equivalent to unsolvated forms and are intended to be encompassed within the scope of the present invention.

In another embodiment, the compositions of the present invention are useful for parenteral administration, such as intravenous (IV) administration or administration into a body cavity or lumen of an organ. The formulations for administration will commonly include a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compositions (e.g., nanoparticles described herein) described herein can be used in combination with one another, with other active agents known to be useful in detecting cancer (e.g. ovarian cancer, bladder cancer, head and neck cancer, brain cancer, breast cancer, lung cancer, cervical cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, bone cancer, spinal cancer, or prostate cancer), or with adjunctive agents that may not be effective alone, but may contribute to the efficacy of the active agent.

In embodiments, the compositions (e.g., nanoparticles described herein) described herein can be co-administered with conventional chemotherapeutic agents including alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, methotrexate, leucovorin, capecitabine, cytarabine, floxuridine, fludarabine, gemcitabine, pemetrexed, raltitrexed, etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, or carboplatin), and the like.

The compositions (e.g., nanoparticles described herein) described herein can also be co-administered with conventional hormonal therapeutic agents including, but not limited to, steroids (e.g., dexamethasone), finasteride, aromatase inhibitors, tamoxifen, and gonadotropin-releasing hormone agonists (GnRH) such as goserelin.

In a further embodiment, the compositions (e.g., nanoparticles described herein) described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

The pharmaceutical compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate.

Formulations suitable for oral administration can comprise: (a) liquid solutions, such as an effective amount of a packaged composition (e.g., nanoparticles described herein) suspended in diluents, e.g., water, saline, or PEG 400; (b) capsules, sachets, or tablets, each containing a predetermined amount of a composition (e.g., nanoparticles described herein), as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise a compositions (e.g., nanoparticles described herein) in a flavor, e.g., sucrose, as well as pastilles including the polypeptide or peptide fragment in an inert base, such as gelatin and glycerin or sucrose and acacia emulsions, gels, and the like, containing, in addition to the polypeptide or peptide, carriers known in the art.

The nanoparticles or agent (e.g., detectable agent) of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which includes an effective amount of a packaged composition (e.g., nanoparticles described herein) with a suppository base. Suitable suppository bases include natural or synthetic triglycerides or paraffin hydrocarbons. In addition, it is also possible to use gelatin rectal capsules which contain a combination of the compositions (e.g., nanoparticles described herein) of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, orally, topically, intraperitoneally, intravesically, or intrathecally. Intraperitoneal administration, parenteral administration, oral administration, and intravenous administration are the preferred methods of administration. The formulations of compositions (e.g., nanoparticles described herein) can be presented in unit-dose or multi-dose sealed containers, such as ampoules and vials.

The pharmaceutical preparation is preferably in unit dosage form. In such form the preparation is subdivided into unit doses containing appropriate quantities of the active component, e.g., nanoparticles. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain compatible therapeutic agents.

In embodiments, the cancer is ovarian cancer, bladder cancer, head and neck cancer, brain cancer, breast cancer, lung cancer, cervical cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, or prostate cancer. In embodiments, the cancer is ovarian cancer, bladder cancer, or head and neck cancer. In embodiments, the cancer is ovarian cancer.

In embodiments, the nanoparticle is administered via intraperitoneal injection, intraurethral injection, or intramuscular injection. In embodiments, the nanoparticle is administered via intraperitoneal injection. In embodiments, the nanoparticle is administered via intraurethral injection. In embodiments, the nanoparticle is administered via intramuscular injection.

### IV. Methods of Use

In an aspect is provided a method of detecting a cancer cell or tumor in a subject including: (a) administering into the peritoneum of the subject a nanoparticle, wherein the nanoparticle includes a detectable agent; and (b) detecting the nanoparticle at the site of the cancer cell or the tumor in the subject; thereby detecting the cancer cell or tumor in the subject.

In embodiments, prior to detecting the nanoparticle (e.g., step b) the method further includes contacting the cancer cell or tumor with the nanoparticle. In embodiments, prior to detecting the nanoparticle (e.g., step b), the method further includes allowing the nanoparticle to the cancer cell or tumor. In embodiments, prior to detecting the nanoparticle (e.g., step b), the method further includes allowing the nanoparticle to migrate to the site of the cancer cell or the tumor.

The site of the cancer cell or the tumor is the space (e.g., area or location) proximal to the cancer cell or tumor or the cancer cell or tumor itself. In embodiments, the site of the cancer cell or the tumor is the peripheral boundary (e.g., cell membrane or peripheral border cells) of the cancer cell or tumor. In embodiments, the site of the cancer cell or the tumor is cell membrane of the cancer cell or a cell of the tumor. In embodiments, the site the tumor is the peripheral cells at the exterior of the tumor or at the boundary (e.g., border) of the tumor. In embodiments the site of the cancer cell or the tumor is the location of contact between the nanoparticle and the cancer cell or tumor. In embodiments, the site is proximal to the cancer cell or tumor. In embodiments, the site is about approximately 0.1, 0.2, 0.5, 1, 2, 3, 4, 5, 10, 15, 20, 25, 30, 40, 50, 60, 70, 80, 90, or about 100 nm from the cancer cell or tumor. In embodiments, the site is about 0.1, 0.2, 0.5, 1, 2, 3, 4, 5, 10, 15, 20, 25, 30, 40, 50, 60, 70, 80, 90, or about 100 μm from the cancer cell or tumor. In embodiments, the site is 0.1, 0.2, 0.5, 1, 2, 3, 4, 5, 6, 7, 8, 9, or about 10 mm from the cancer cell or tumor.

In embodiments, the site of the cancer cell is a cancer cell. In embodiments the site of the tumor is a tumor. In embodiments, the site of the cancer cell or the tumor is a macrophage proximal to the tumor or cancer cell. In embodiments, the site is a tumor-associated macrophage.

In embodiments, where the site of the cancer cell or the tumor is a macrophage, the macrophage expresses CD45, CD11b, and/or f4/80. In embodiments, the macrophage is a tumor-associated macrophage (e.g., a macrophage located in close proximity to or within a neoplasm). In embodiments, the macrophage is a Type I or Type II macrophage. In embodiments, the macrophage is a Type II macrophage.

In embodiments, the method of detecting a cancer cell in a subject includes: (a) administering into the peritoneum of the subject a nanoparticle, wherein the nanoparticle includes a detectable agent; and (b) detecting the nanoparticle at the site of the cancer cell in the subject; thereby detecting the cancer cell in the subject. In embodiments, the method of detecting a tumor in a subject including: (a) administering into the peritoneum of the subject a nanoparticle, wherein the nanoparticle includes a detectable agent; and (b) detecting the nanoparticle at the site of the tumor in the subject; thereby detecting the tumor in the subject.

In embodiments, the method of detecting a cancer cell in a subject includes: (a) administering into the peritoneum of the subject a silica nanoparticle, wherein the silica nanoparticle includes a detectable agent; and (b) detecting the silica nanoparticle at the site of the cancer cell in the subject; thereby detecting the cancer cell in the subject. In embodiments, the method of detecting a tumor in a subject including: (a) administering into the peritoneum of the subject a silica nanoparticle, wherein the silica nanoparticle includes a detectable agent; and (b) detecting the silica nanoparticle at the site of the tumor in the subject; thereby detecting the tumor in the subject.

In embodiments, the method of detecting a cancer cell in a subject includes: (a) administering into the peritoneum of the subject an unmodified silica nanoparticle, wherein the unmodified silica nanoparticle includes a detectable agent; and (b) detecting the unmodified silica nanoparticle at the site of the cancer cell in the subject; thereby detecting the cancer cell in the subject. In embodiments, the method of detecting a tumor in a subject including: (a) administering into the peritoneum of the subject an unmodified silica nanoparticle, wherein the unmodified silica nanoparticle includes a detectable agent; and (b) detecting the unmodified silica nanoparticle at the site of the tumor in the subject; thereby detecting the tumor in the subject.

In embodiments, the cancer cell is an ovarian cancer cell, bladder cancer cell, head and neck cancer cell, brain cancer cell, breast cancer cell, lung cancer cell, cervical cancer cell, bone cancer cell, spinal cancer cell, liver cancer cell, colorectal cancer cell, pancreatic cancer cell, glioblastoma cell, neuroblastoma cell, rhabdomyosarcoma cell, osteosarcoma cell, renal cancer cell, renal cell carcinoma, non-small cell lung cancer cell, uterine cancer cell, testicular cancer cell, anal cancer cell, bile duct cancer cell, biliary tract cancer cell, gastrointestinal carcinoid tumor cell, esophageal cancer cell, gall bladder cancer cell, appendix cancer cell, small intestine cancer cell, stomach (gastric) cancer cell, urinary bladder cancer cell, genitourinary tract cancer cell, endometrial cancer cell, nasopharyngeal cancer cell, head and neck squamous cell carcinoma, or prostate cancer cell. In embodiments, the cancer cell is an ovarian cancer cell.

In embodiments, the cancer cell forms part of a tumor. In embodiments, the tumor is an ovarian tumor, bladder tumor, pancreatic tumor, colorectal tumor, gastric tumor, bone tumor, spinal tumor, or liver tumor. In embodiments, the tumor is an ovarian tumor.

In embodiments, the tumor includes stromal cells, immune cells, proteins, and extracellular matrix generated by stromal or immune cells. In embodiments, the tumor includes macrophage cells. In embodiments, immune cells (e.g., macrophage cells), stromal cells, proteins associate with the immune cells, proteins associated with the stromal cells, and the extracellular matrix generated from immune cells and stromal cells forms part of a tumor.

In embodiments, the method of detecting a cancer cell or tumor in a subject includes: (a) administering into the peritoneum (e.g., via intraperitoneal administration) of the subject a nanoparticle, as described herein including embodiments, wherein the nanoparticle includes a detectable agent; (b) allowing the nanoparticle to contact a cancer cell or tumor within the subject; and (c) detecting the nanoparticle in contact with the cancer cell or tumor thereby detecting the cancer cell in the subject.

In embodiments, the method of detecting a cancer cell or tumor in a subject includes: (a) administering into the subject a nanoparticle, as described herein including embodiments, wherein the nanoparticle includes a detectable agent; (b) allowing the nanoparticle to contact a cancer cell or tumor within the subject; and (c) detecting the nanoparticle in contact with the cancer cell thereby detecting the cancer cell in the subject.

In embodiments, the method of detecting a cancer cell or tumor in a subject includes: (a) administering into the subject a nanoparticle-cell construct, as described herein including embodiments, wherein the nanoparticle includes a detectable agent; (b) allowing the nanoparticle-cell construct to contact a cancer cell or tumor within the subject; and (c) detecting the nanoparticle-cell construct in contact with the cancer cell thereby detecting the cancer cell in the subject.

In embodiments, the method of identifying a cell in a patient, includes: (a) contacting the cell with a nanoparticle, as described herein including embodiments, wherein the nanoparticle includes a detectable agent; (b) detecting the presence of the detectable agent contacting the cell; and thereby identifying the cell.

In embodiments, the method includes: (a) administering a plurality of unmodified silica nanoparticles via intraperitoneal injection; (b) contacting the cell with the unmodified silica nanoparticles, wherein the unmodified silica nanoparticles include a detectable agent; (c) detecting the presence of the detectable agent included in the nanoparticle contacting the cell; and thereby identifying the cell.

In embodiments, the method of detecting a cancer cell or tumor in a subject includes: (a) administering into the subject a nanoparticle, as described herein including embodiments, wherein the nanoparticle includes a detectable agent; (b) allowing the nanoparticle to contact a macrophage within the subject; and (c) detecting the nanoparticle in contact with the macrophage thereby detecting the tumor in the subject.

In embodiments, the nanoparticle, cell, or construct is administered via intraperitoneal injection, intratumoral injection, intraurethral injection, or intramuscular injection. In embodiments, the nanoparticle, cell, or construct is administered via intraperitoneal injection. In embodiments, the nanoparticle, cell, or construct is administered via intraurethral injection. In embodiments, the nanoparticle, cell, or construct is administered via intramuscular injection. In embodiments, the nanoparticle, cell, or construct is administered via intratumoral injection. In embodiments, the nanoparticle is administered via intraperitoneal injection.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### V. Embodiments

Embodiment P1. A method of detecting a cancer cell or tumor in a subject comprising:
a) administering into the peritoneum of said subject a nanoparticle, wherein the nanoparticle comprises a detectable agent;
b) allowing said nanoparticle to contact a cancer cell or tumor within said subject; and
c) detecting the nanoparticle in contact with said cancer cell or tumor thereby detecting the cancer cell or tumor in said subject.

Embodiment P2. The method of embodiment P1, wherein the nanoparticle is a silica nanoparticle.

Embodiment P3. The method of one of embodiments P1 or P2, wherein the nanoparticle is an unmodified silica nanoparticle.

Embodiment P4. The method of one of embodiments P1 or P3, wherein the nanoparticle is covalently attached to one or more nanoparticle substituents, wherein said nanoparticle substituents are independently:

-L²-X¹—R³; i)

-L²-X¹-L¹-X³; or ii)

-L²-X³; iii)
wherein
X¹ is a bioconjugate linker or a bond;
X³ is a bioconjugate reactive group;
L¹ is a independently a polymeric linker;
L² is independently a bond, —NR^{1a}—, —O—, —S—, —C(O)—, —C(O)O—, —S(O)—, —S(O)₂—, —NR^{1a}C(O)—, —C(O)NR^{1b}—, —C(O)(CH₂)_{z1}—, —NR^{1a}C(O)O—, —NR^{1a}C(O)NR^{1b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{1a} and R^{1b} are independently hydrogen,
   halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
the symbol z1 is an integer from 1 to 10; and
R³ is a polymeric moiety.

Embodiment P5. The method of embodiment P4, wherein R³ is a polyethylene glycol moiety.

Embodiment P6. The method of embodiments P4 or P5, wherein the bioconjugate reactive group is —NH₂, —COOH, —N-hydroxysuccinimide, or maleimide.

Embodiment P7. The method of one of embodiments P4 to P6 wherein the nanoparticle is covalently attached to a plurality of nanoparticle substituents of the formula (i) and a plurality of nanoparticle substituents of the formula (ii) in a ratio of about 50:50 to about 80:20.

Embodiment P8. The method of one of embodiments P4 to P6 wherein the nanoparticle is covalently attached to a plurality of nanoparticle substituents of the formula (ii) and a plurality of nanoparticle substituents of the formula (iii) in a ratio of about 50:50 to about 80:20.

Embodiment P9. The method of any one of embodiments P4 to P6 wherein the nanoparticle is covalently attached to a plurality of nanoparticle substituents of the formula (i) and a plurality of nanoparticle substituents of the formula (iii) in a ratio of about 50:50 to about 80:20.

Embodiment P10. The method of any one of embodiments P4 to P9, wherein each L¹ is independently a linear polymeric linker or branched polymeric linker.

Embodiment P11. The method of any of embodiments P1 to P10, wherein the detectable agent is a radioisotope, fluorophore, electron-dense reagent, enzyme, biotin, paramagnetic agent, or magnetic agent.

Embodiment P12. The method of any of embodiments P1 to P10, wherein the detectable agent is a fluorophore.

Embodiment P13. The method of any one of embodiments P1 to P12, wherein the detectable agent is a fluorophore having an emission wavelength from about 495 nm to about 570 nm.

Embodiment P14. The method of any one of embodiments P1 to P12, wherein the detectable agent is a fluorophore having an emission wavelength from about 570 nm to about 620 nm.

Embodiment P15. The method of any one of embodiments P1 to P12, wherein the detectable agent is a fluorophore having an emission wavelength from about 620 nm to about 650 nm.

Embodiment P16. The method of any one of embodiments P1 to P12, wherein the detectable agent is a fluorophore having an emission wavelength from about 710 nm to about 850 nm.

Embodiment P17. The method of any one of embodiments P1 to P12, wherein the detectable agent is a fluorophore having an emission wavelength from about 850 nm to about 1350 nm.

Embodiment P18. The method of any one of embodiments P1 to P12, wherein the detectable agent comprises a cyanine, heptamethine, xanthene, rhodamine, fluorescein, boron-dipyrromethene, boron dipyridyl, naphthalene, coumarin, acridine, acridinium, tetrapyrrole, tetraphenylethene, oxazine, pyrene, oxadiazole, subphthalocyanine, carbopyrinin, benzopyrinium, or phthalocyanine.

Embodiment P19. The method of one of embodiments P1 to P18, wherein the average longest dimension of the nanoparticle is from about 10 nm to about 600 nm.

Embodiment P20. The method of one of embodiments P1 to P18, wherein the average longest dimension of the nanoparticle is from about 100 nm to about 400 nm.

Embodiment P21. The method of one of embodiments P1 to P18, wherein the average longest dimension of the nanoparticle is from about 170 nm to 270 nm.

Embodiment P22. The method of one of embodiments P1 to P21, wherein the nanoparticle further comprises a stabilizing agent.

Embodiment P23. The method of embodiment P22, wherein the stabilizing agent is a surfactant or a polymer.

Embodiment P24. The method of one of embodiments P1 to P23, wherein the cancer cell is an ovarian cancer cell, bladder cancer cell, head and neck cancer cell, brain cancer cell, breast cancer cell, lung cancer cell, cervical cancer cell, bone cancer cell, spinal cancer cell, liver cancer cell, colorectal cancer cell, pancreatic cancer cell, glioblastoma cell, neuroblastoma cell, rhabdomyosarcoma cell, osteosarcoma cell, renal cancer cell, renal cell carcinoma, non-small cell lung cancer cell, uterine cancer cell, testicular cancer cell, anal cancer cell, bile duct cancer cell, biliary tract cancer cell, gastrointestinal carcinoid tumor cell, esophageal cancer cell, gall bladder cancer cell, appendix cancer cell, small intestine cancer cell, stomach (gastric) cancer cell, urinary bladder cancer cell, genitourinary tract cancer cell, endometrial cancer cell, nasopharyngeal cancer cell, head and neck squamous cell carcinoma, or prostate cancer cell.

Embodiment P25. The method of one of embodiments P1 to P24, wherein the cancer cell is part of a tumor.

Embodiment P26. The method of embodiment P25, wherein the tumor is an ovarian tumor, bladder tumor, pancreatic tumor, colorectal tumor, gastric tumor, bone tumor, spinal tumor, or liver tumor.

### VI. Additional Embodiments

Embodiment 1. A method of detecting a cancer cell or tumor in a subject comprising: (a) administering into the peritoneum of said subject a nanoparticle, wherein the nanoparticle comprises a detectable agent; and (b) detecting said nanoparticle at the site of said cancer cell or said tumor in said subject; thereby detecting the cancer cell or tumor in said subject.

Embodiment 2. The method of embodiment 1, wherein the nanoparticle is a silica nanoparticle.

Embodiment 3. The method of one of embodiments 1 or 2, wherein the nanoparticle is an unmodified silica nanoparticle.

Embodiment 4. The method of one of embodiments 1 or 2, wherein the nanoparticle is covalently attached to one or more nanoparticle substituents, wherein said nanoparticle substituents are independently:

-L²-X¹—R³; i)

-L²-X¹-L¹-X³; or ii)

-L²-X³; iii)
wherein
X¹ is a bioconjugate linker or a bond;
X³ is a bioconjugate reactive group;
L¹ is a polymeric linker;
L² is independently a bond, —NR^{1a}—, —O—, —S—, —C(O)—, —C(O)O—, —S(O)—, —S(O)₂—, —NR^{1a}C(O)—, —C(O)NR^{1b}—, —C(O)(CH₂)_{z1}—, —NR^{1a}C(O)NR^{1a}C(O)NR^{1b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{1a} and R^{1b} are independently hydrogen,
   halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
the symbol z1 is an integer from 1 to 10; and
R³ is a polymeric moiety.

Embodiment 5. The method of embodiment 4, wherein R³ is a polyethylene glycol moiety.

Embodiment 6. The method of embodiments 4 or 5, wherein the bioconjugate reactive group is —NH₂, —COOH,

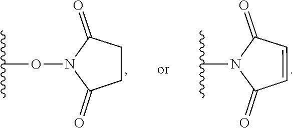 [SMILES: *ON1C(=O)CCC1=O ; *N1C(=O)C=CC1=O]

Embodiment 7. The method of one of embodiments 4 to 6 wherein the nanoparticle is covalently attached to a plurality of nanoparticle substituents of the formula (i) and a plurality of nanoparticle substituents of the formula (ii) in a ratio of about 50:50 to about 80:20.

Embodiment 8. The method of one of embodiments 4 to 6 wherein the nanoparticle is covalently attached to a plurality of nanoparticle substituents of the formula (ii) and a plurality of nanoparticle substituents of the formula (iii) in a ratio of about 50:50 to about 80:20.

Embodiment 9. The method of any one of embodiments 4 to 6 wherein the nanoparticle is covalently attached to a plurality of nanoparticle substituents of the formula (i) and a plurality of nanoparticle substituents of the formula (iii) in a ratio of about 50:50 to about 80:20.

Embodiment 10. The method of any one of embodiments 4 to 9, wherein each L¹ is independently a linear polymeric linker or branched polymeric linker.

Embodiment 11. The method of any of embodiments 1 to 10, wherein the detectable agent is a radioisotope, fluorophore, electron-dense reagent, enzyme, biotin, paramagnetic agent, or magnetic agent.

Embodiment 12. The method of any of embodiments 1 to 10, wherein the detectable agent is a fluorophore.

Embodiment 13. The method of any one of embodiments 1 to 12, wherein the detectable agent is a fluorophore having a maximum emission wavelength from about 495 nm to about 570 nm.

Embodiment 14. The method of any one of embodiments 1 to 12, wherein the detectable agent is a fluorophore having a maximum emission wavelength from about 570 nm to about 620 nm.

Embodiment 15. The method of any one of embodiments 1 to 12, wherein the detectable agent is a fluorophore having a maximum emission wavelength from about 620 nm to about 650 nm.

Embodiment 16. The method of any one of embodiments 1 to 12, wherein the detectable agent is a fluorophore having a maximum emission wavelength from about 710 nm to about 850 nm.

Embodiment 17. The method of any one of embodiments 1 to 12, wherein the detectable agent is a fluorophore having a maximum emission wavelength from about 850 nm to about 1350 nm.

Embodiment 18. The method of any one of embodiments 1 to 12, wherein the detectable agent comprises cyanine, heptamethine, xanthene, rhodamine, fluorescein, boron-dipyrromethene, boron dipyridyl, naphthalene, coumarin, acridine, acridinium, tetrapyrrole, tetraphenylethene, oxazine, pyrene, oxadiazole, subphthalocyanine, carbopyrinin, benzopyrinium, or phthalocyanine.

Embodiment 19. The method of one of embodiments 1 to 18, wherein the average longest dimension of the nanoparticle is from about 10 nm to about 1000 nm.

Embodiment 20. The method of one of embodiments 1 to 18, wherein the average longest dimension of the nanoparticle is from about 10 nm to about 600 nm.

Embodiment 21. The method of one of embodiments 1 to 18, wherein the average longest dimension of the nanoparticle is from about 100 nm to about 400 nm.

Embodiment 22. The method of one of embodiments 1 to 18, wherein the average longest dimension of the nanoparticle is from about 170 nm to 270 nm.

Embodiment 23. The method of one of embodiments 1 to 21, wherein the nanoparticle further comprises a stabilizing agent.

Embodiment 24. The method of embodiment 22, wherein the stabilizing agent is a surfactant or a polymer.

Embodiment 25. The method of one of embodiments 1 to 23, wherein the cancer cell is an ovarian cancer cell, bladder cancer cell, head and neck cancer cell, brain cancer cell, breast cancer cell, lung cancer cell, cervical cancer cell, bone cancer cell, spinal cancer cell, liver cancer cell, colorectal cancer cell, pancreatic cancer cell, glioblastoma cell, neuroblastoma cell, rhabdomyosarcoma cell, osteosarcoma cell, renal cancer cell, renal cell carcinoma cell, non-small cell lung cancer cell, uterine cancer cell, testicular cancer cell, anal cancer cell, bile duct cancer cell, biliary tract cancer cell, gastrointestinal carcinoid tumor cell, esophageal cancer cell, gall bladder cancer cell, appendix cancer cell, small intestine cancer cell, stomach (gastric) cancer cell, urinary bladder cancer cell, genitourinary tract cancer cell, endometrial cancer cell, nasopharyngeal cancer cell, head and neck squamous cell carcinoma cell, or prostate cancer cell.

Embodiment 26. The method of one of embodiments 1 to 24, wherein the cancer cell is part of a tumor.

Embodiment 27. The method of embodiment 26, wherein the tumor is an ovarian tumor, bladder tumor, pancreatic tumor, colorectal tumor, gastric tumor, bone tumor, spinal tumor, or liver tumor.

Embodiment 28. A nanoparticle-cell construct comprising a silica nanoparticle covalently attached to a protein through a covalent linker, said covalent linker having the formula:

-L²-X¹-L¹-X²-L³; (Ia) or

-L²-X²-L³; (Ib)
wherein
X¹ and X² are independently a bioconjugate linker or a bond, wherein at least one of X¹ or X² is a bioconjugate linker;
L¹ is independently a polymeric linker;
L² is independently a bond, —NR^{1a}—, —O—, —S—, —C(O)—, —C(O)O—, —S(O) S(O)₂—, —NR^{1a}C(O)—, —C(O)NR^{1b}—, —C(O)(CH₂)_{z1}—, —NR^{1a}C(O)O—, —NR^{1a}C(O)NR^{1b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is independently a bond, —NR^{2a}—, —O—, —C(O)—, —C(O)O—, —S(O)—, —S(O)₂—, —NR^{2a}C(O)—, —C(O)NR^{2b}—, —C(O)(CH₂)_{z2}—, —NR^{2a}C(O)NR^{2b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{1a}, R^{2a}, R^{1b}, and R^{2b} are independently hydrogen,
   halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
the symbols z1 and z2 are independently an integer from 1 to 10.

Embodiment 29. The nanoparticle-cell construct of embodiment 28, wherein the silica nanoparticle comprises a detectable agent.

Embodiment 30. The nanoparticle-cell construct of embodiment 29, wherein the detectable agent is a fluorophore.

### Examples

A. Synthesis of Nanoparticles

In a standard reaction, 40 mL tetraethyl orthosilicate (TEOS) were added to a microemulsion system that contains a mixture of 7.7 mL cyclohexane, 2 mL Triton X-100, 1.6 mL hexanol, and 0.34 mL MilliQ water. This mixture was stirred at 400 rpm for 5 h, at room temperature, followed by the addition of 100 μL aqueous ammonia. This reaction mixture was stirred at 400 rpm for 16 h, at room temperature. To prepare the fluorophore for silica nanoparticle incorporation, 35 μmol of amine-reactive fluorophore (NETS- or TFP-activated dyes) and 35 μmol of (3-Aminopropyl)triethoxysilane was added to 100 μL of absolute EtOH and shaken overnight at room temperature. The following day, the mixture containing APS-fluorophore adduct and 20 μL of aqueous ammonia was added to the silica microemulsion system and it was stirred for another 16 hr at room temperature. Upon reaction completion, the mixture was transferred to a 50 mL tube and EtOH was used to quench the NPs out of solution. Fluorescently-labeled SiNPs were collected by centrifugation (3220 g, 10 min). The supernatant was discarded, the NP pellet was redispersed in 2 mL EtOH and transferred to a 2 mL eppendorf. SiNPs were washed via repeated centrifugation by 2 more times with EtOH and 3 more times with MilliQ water (21,000 g, 1.5 min). The NP solution was sonicated in between washes to assist their redispersion back into solution. After the final wash, SiNPs were dispersed in MilliQ water and stored at 4° C.

A 25 mL round bottom flask with a magnetic stirring bar was flushed with nitrogen for 10 minutes. A dispersion of red silica nanoparticles (500 nm, 3.8×10″ NPs) in 4 mL ethanol was added to the flask under nitrogen followed by the addition of 0.67 mL of aqueous ammonia. The final NP concentration was 10 g/L in the solution mixture with a final ammonia concentration of 4 vol. %. (3-Aminopropyl)triethoxysilane (APTES, 1 μL) in 0.33 mL of EtOH was then added to the reaction mixture and it was stirred at room temperature overnight. The following day, the reaction was refluxed at 85° C. while stirring for 2 h. The resulting NPs in the dispersion were collected and washed by repeated centrifugation at 21,000 g, 1 min (3 washes in EtOH, followed by 3 washes in MilliQ water). The amount of APTES was calculated under the assumption that each APTES molecule takes up 0.6 nm² on the NP surface. To ensure the complete conversion of the hydroxyl groups to amine groups on the NP surface, a 7-fold excess of APTES was used in the reaction. SiNP—NH₂ was redispersed in MilliQ water and stored at 4° C.; see FIG. 1.

Functionalizing terminal NH₂ with maleimide. A water dispersion of SiNP—NH₂ containing 1.9×10¹¹ NPs was exchanged to PBS solution by 3 repeated centrifugation cycles at 21,000 g, 1 min in PBS. A 25-fold molar excess of sulfo-SMCC solution in PBS was added to the SiNP—NH₂ and the mixture was shaken at 37° C. for 1 hr. To remove the salts and excess sulfo-SMCC, SiNPs were pelleted and washed 3 times with MilliQ water by centrifugation (21,000 g, 1 min). The resulting SiNP-Mal particles were redispersed in MilliQ water and stored at 4° C.; see FIG. 1.

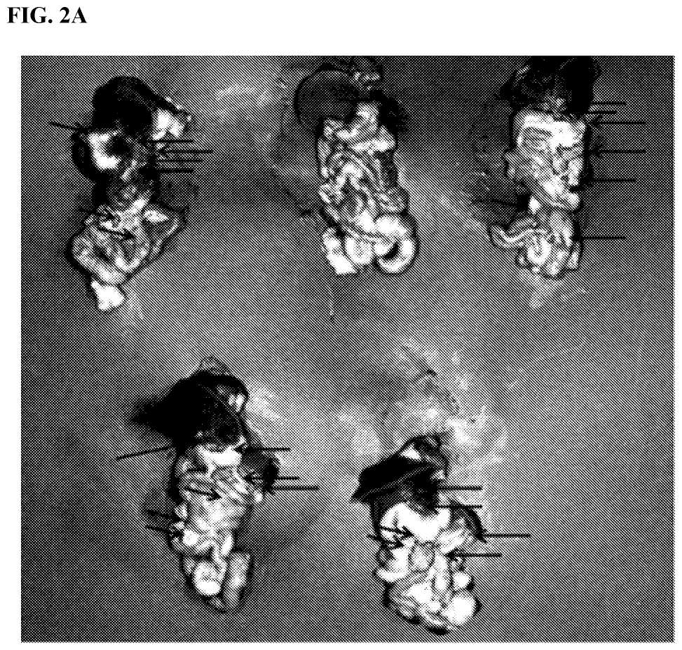

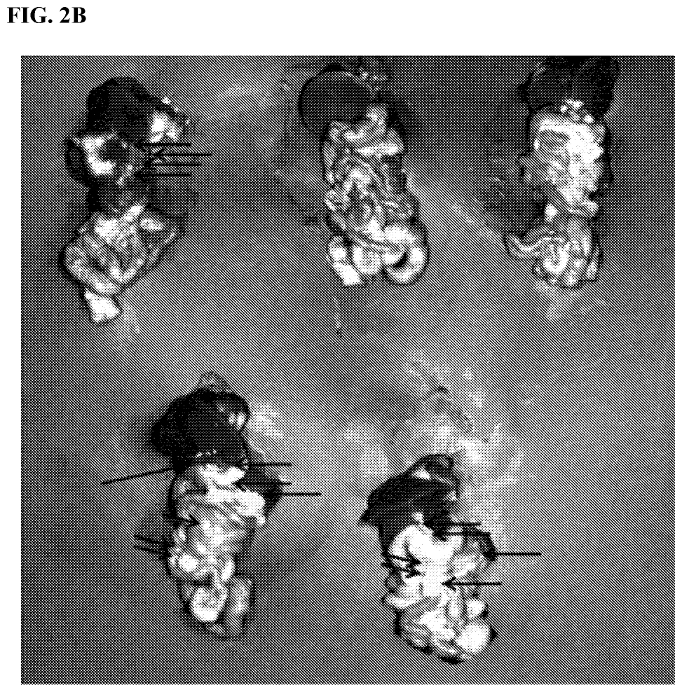

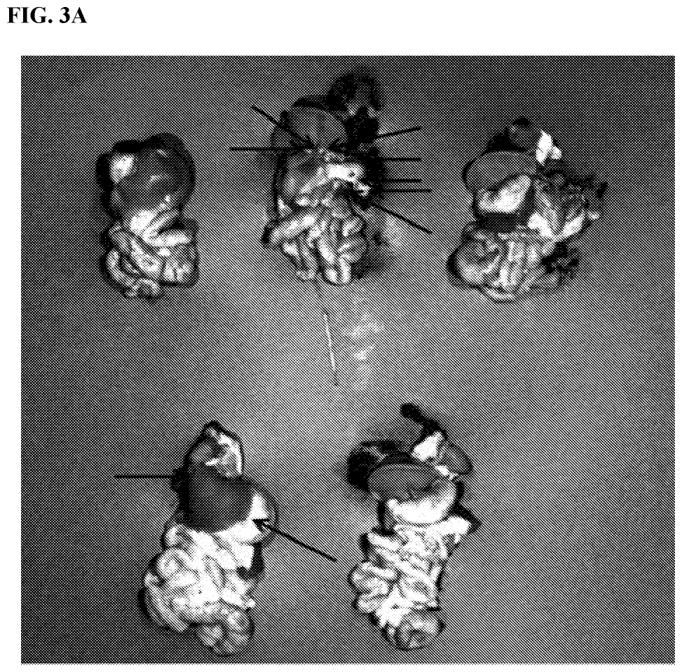

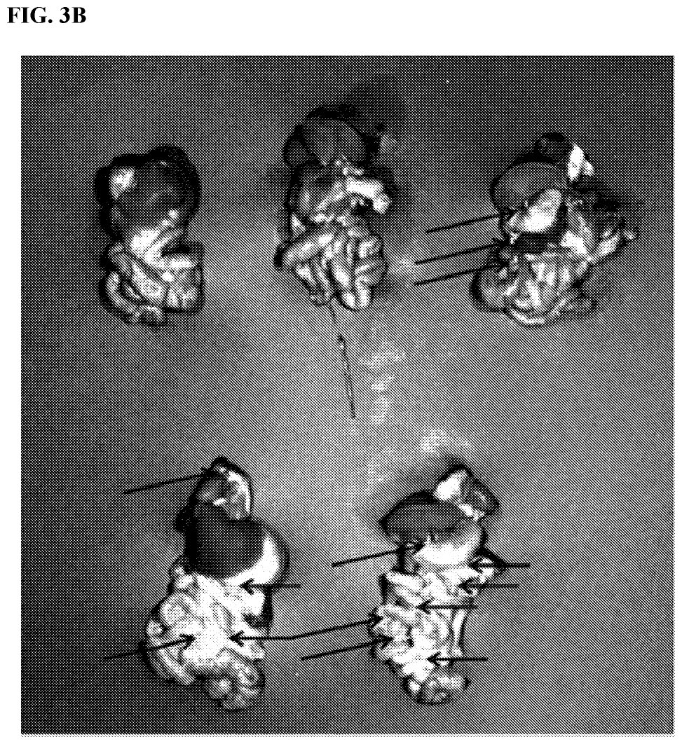

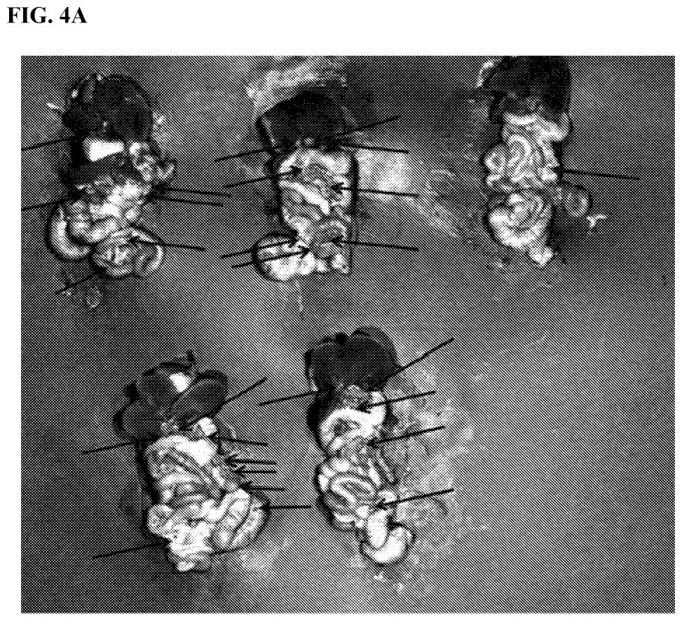

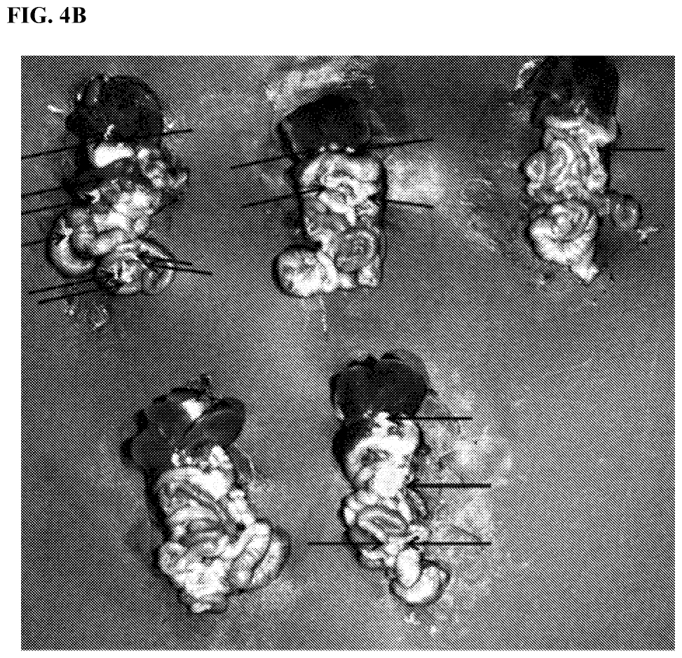

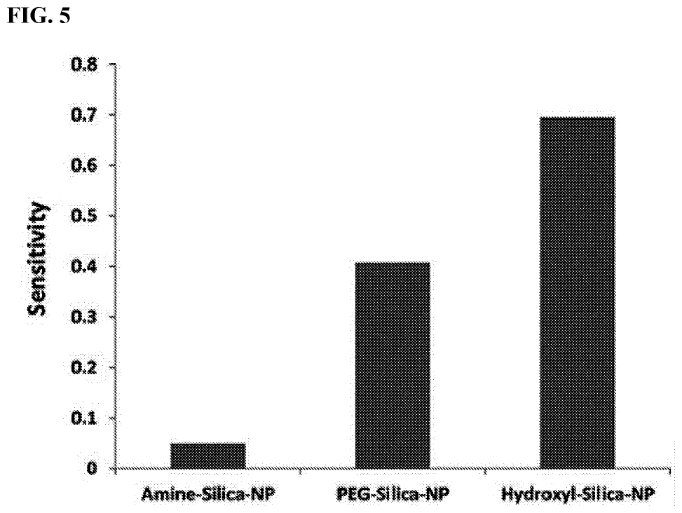

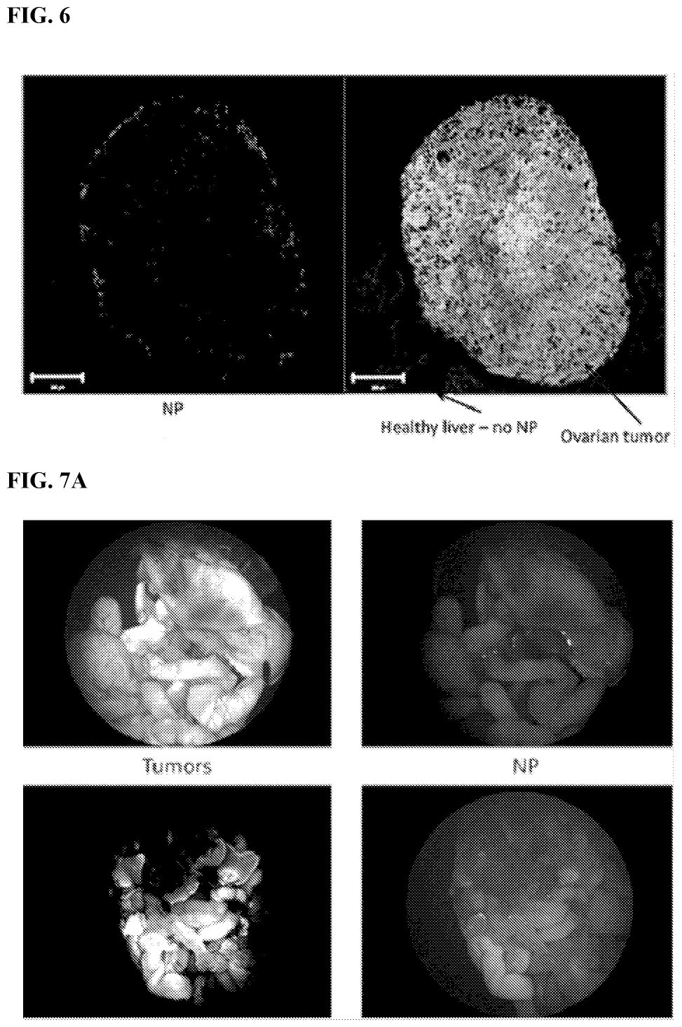

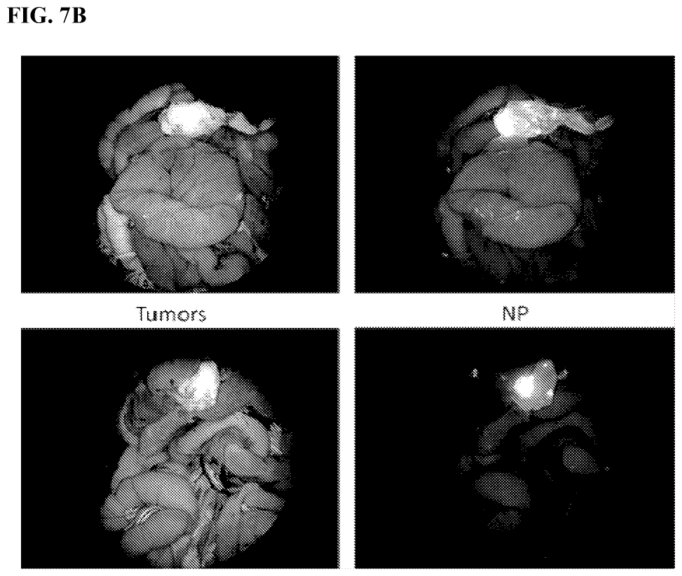

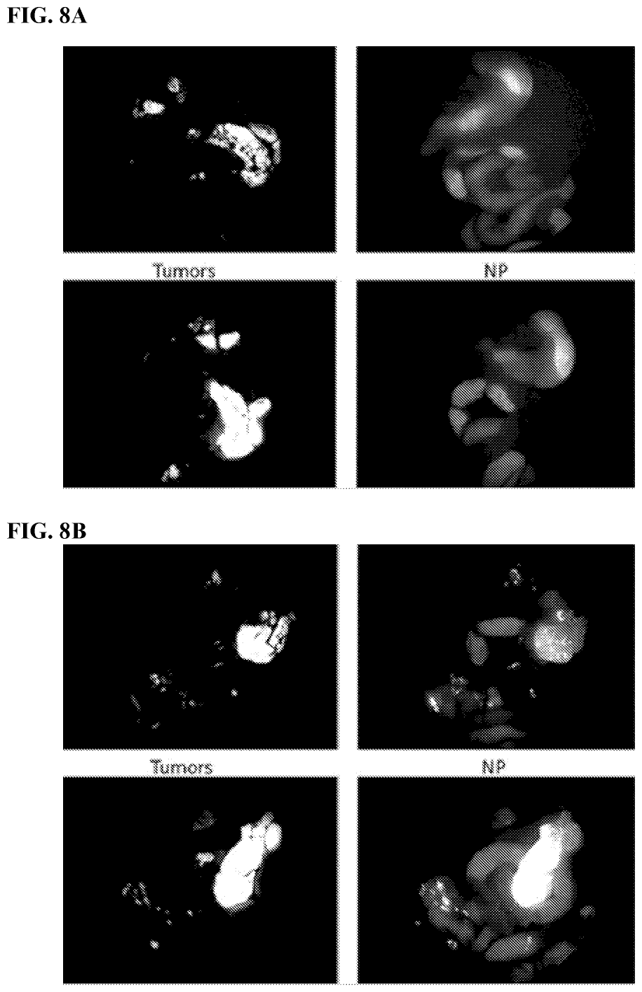

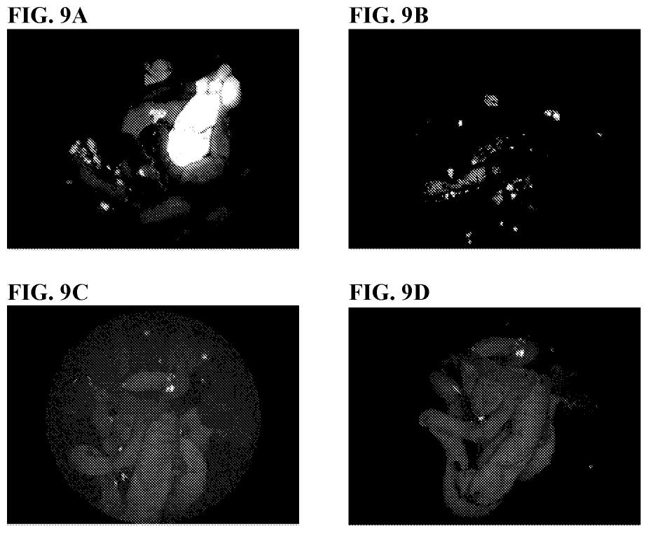

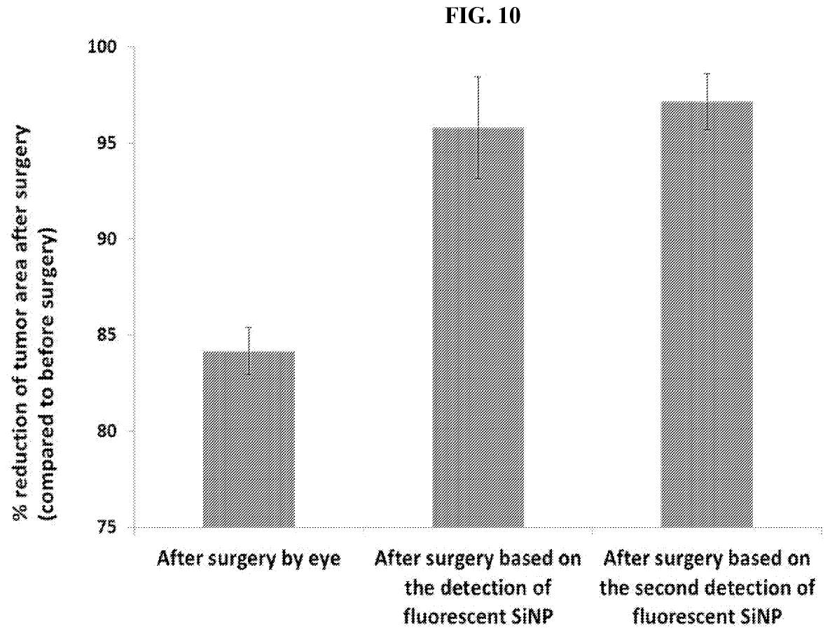

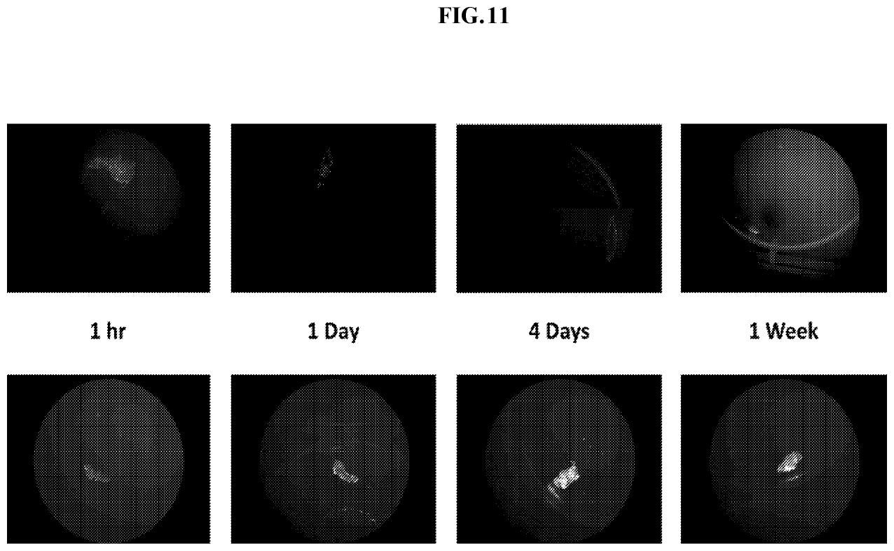

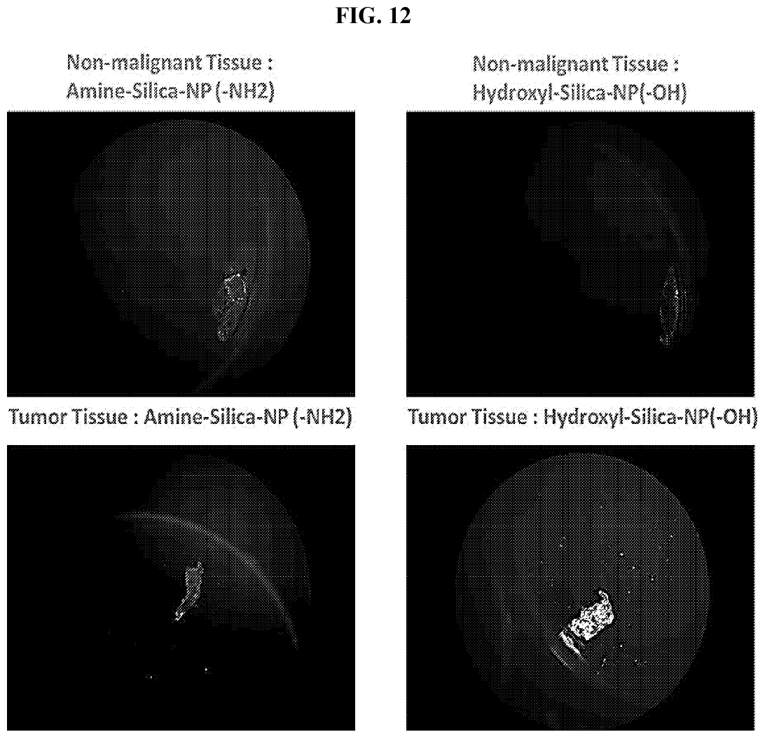

Cell labeling study for SEM images, as observed in FIG. 2. 1. Take 2 small, circular cover glasses, sterilize them by soaking them in absolute EtOH overnight. 2. Take the cover glasses out of EtOH using tweezers, once dried, flame them over the fire. 3. Put each cover glass in a 24-well plate well, close the lid, leave the plate under UV light for 10 min. 4. Add 0.33M NSCs in 0.5 mL of media to each well with cover glass. Let the cells adhere overnight. 5. Next day, remove the old media. Cells were washed once with PBS. 6. Treat cells with NPs (SiNP—OH, 1 μL; SiNP-Mal, 2 μL) in 0.5 mL DMEM media without amine or free thiol groups, incubate at 4° C. for 30 min. 7. Remove the media, wash cells once with PBS 8. Fix the cells with 1 mL of 2% glutaraldehyde in each well, leave samples in the fixing solution for 1 hr at r.t. 9. Samples were dried and stained for SEM imaging.

TABLE 1
Library of polyethylene glycol (PEG)
used to coat the nanoparticle surface.

| Linear PEGs | Branched PEGs |
|---|---|
| mPEG₄₀₀-SH | TFP-(m-dPEG₁₁)₃ |
| mPEG₁₀₀₀-SH | NHS-(m-dPEG₂₄)₃ |
| mPEG₁₀₀₀-NHS | |
| mPEG₂₀₀₀-SH | |
| mPEG₂₀₀₀-NHS | |
| Mal-PEG₂₀₀₀-NHS | |
| Mal-PEG₃₄₀₀-NHS | |
| mPEG₅₀₀₀-SH | |
| Mal-PEG₅₀₀₀-NHS | |

Functionalizing silica nanoparticles terminated with -Mal with linear PEGs selected from Table 1. SiNP-Mal in MilliQ was washed 3 times with PBS to convert their solvent to PBS followed by the addition of a PEG-SH solution in PBS. The mixture was placed in a shaker and incubated at 37° C. overnight. It was assumed that each maleimide group on the NP surface takes up 0.6 nm² and each maleimide functional group reacts with one thiol group on the PEG-SH molecules. To maximize PEG coverage on the NP surface, 10-fold molar excess of PEG-SH to the number of maleimide groups on the SiNP surface was used in the reaction. Upon reaction completion, PEGylated SiNPs were collected and washed by repeated centrifugation at 21,000 g for 1 min (3 times with MilliQ water). PEGylated SiNPs were resuspended in MilliQ water and stored at 4° C.

Functionalizing silica nanoparticles terminated with NH₂ with branched PEGs selected from Table 1. SiNP—NH₂ in MilliQ was washed 3 times with PBS to convert their solvent to PBS followed by the addition of a TFP-(PEG₁₁)₃ or N-Hydroxysuccinimide-(PEG₂₄)₃ solution in PBS. Note N-Hydroxysuccinimide is alternatively written as NHS. The mixture was placed in a shaker and incubated at 37° C. overnight. It was assumed that each amine group NH₂— on the NP surface takes up 0.6 nm² and each amine functional group reacts with one activated ester group (TFP- or N-Hydroxysuccinimide-) on the branched PEG molecules. To maximize PEG coverage on the NP surface, 10-fold molar excess of PEG to the number of amine groups on the SiNP surface was used in the reaction. Upon reaction completion, PEGylated SiNPs were collected and washed by repeated centrifugation at 21,000 g for 1 min (3 times with MilliQ water). PEGylated SiNPs were resuspended in MilliQ water and stored at 4° C.

Functionalizing silica nanoparticles terminated with NH₂ with functionalized-PEGs. SiNP—NH₂ in MilliQ was washed 3 times with PBS to convert their solvent to PBS followed by the addition of a mixture of TFP-(PEG₁₁)₃ and N-Hydroxysuccinimide-PEG-Mal solution in PBS. The mixture was placed in a shaker and incubated at 37° C. overnight. It was assumed that each maleimide group on the NP surface takes up 0.6 nm² and each maleimide functional group reacts with one thiol group on the PEG-SH molecules. To maximize PEG coverage on the NP surface, 10-fold molar excess of Mal-PEG-NHS to the number of amine groups on the SiNP surface was used in the reaction. Upon reaction completion, PEGylated SiNPs were collected and washed by repeated centrifugation at 21,000 g for 1 min (3 times with MilliQ water). PEGylated SiNPs were resuspended in MilliQ water and stored at 4° C.

Additional synthetic routes are outlined in Scheme 1 below, with one or two reactive groups shown for clarity:

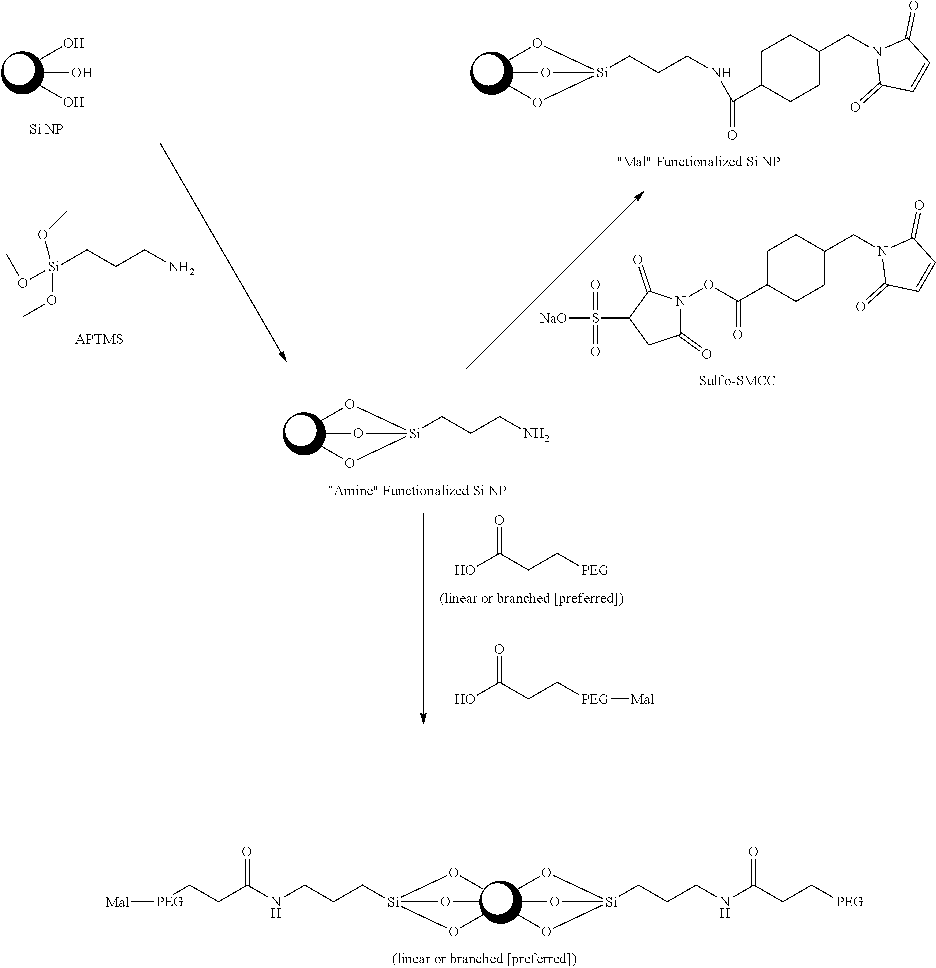

In embodiments, a preferred polymeric linker is

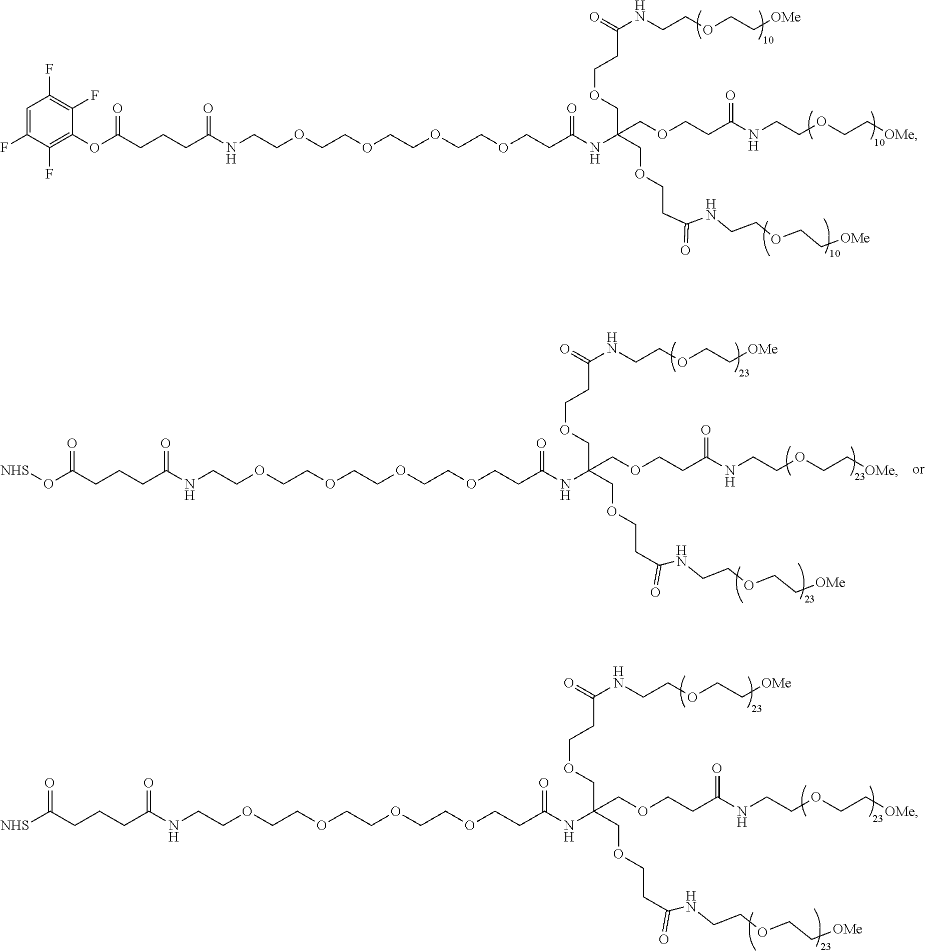

wherein NHS is N-hydroxysuccinimide.

When comparing the size of the PEGs, PEG₁₀₀₀ vs. PEG₂₀₀₀, (PEG₁₁)₃ vs. (PEG₂₄)₃, it appears that shorter PEGs perform better at reducing the non-specific binding of SiNPs to cells as cells had lower level of red fluorescence. When comparing the structure of the PEGs, linear vs. branched, branched PEGs, (PEG₁₁)₃ and (PEG₂₄)₃ work better than the linear PEGs, PEG₁₀₀₀ and PEG₂₀₀₀. Overall, of those tested, (PEG₁₁)₃ is preferred at preventing non-specific binding of the SiNPs to NSCs.

Cell labeling studies. 1. NSCs were plated in 6-well plate. 2. At full confluency, old media was removed and NSCs were washed once with PBS. 3. 1.5 mL of fresh media was added into each well and NSCs were treated with equal amount of bare SiNPs (SiNP—OH, SiNP-Mal) or PEGylated NPs (SiNP-PEG₄₀₀, SiNP-PEG₂₀₀₀, SiNP-PEG₅₀₀₀) for 30 min, at 37° C. 4. Once the incubation was over, the media was removed and NSCs were washed once with PBS. 5. NSCs were then trypsinized and collected by centrifugation (850 g, 3 min). 6. Half of the NSCs were resuspended in PBS and their red fluorescence at 568 nm was measured by a flow cytometer.

NSCs treated with bare SiNPs have very high level of red fluorescence, indicating high level of binding and/or internalization of bare SiNPs to the cells. NSCs treated with PEGylated SiNPs have reduced binding and/or internalization of SiNPs to the cells.

Replating studies. After NSCs treated with SiNPs, half of the trypsinized cells were replated in a 6-well plate. Bright field images were taken after 24 hours of replating the cells. After replating, NSCs treated with bare SiNPs had the NPs around their perinuclear space, an indicator of NP internalization by endocytosis. The PEGylated NPs didn't have many NPs left on the NSCs, likely due to the changed properties of PEGylated surface on the NPs (i.e. less sticky). After trypsinization and replating, there were not many of them left on the cell. For the residual PEG-NPs on the cells, not many of them were in the perinuclear space. This could be due to delayed endocytosis.

Identifying the preferred ratio of non-functionalized PEG:functionalized PEG. NSCs were labeled with PEGylated SiNPs at both 4° C. and 37° C. for 30 min, and only at 37° C. for 30 min. At the composition of 80%:20%, the amount of Mal-PEG on the NP surface was low and NSCs were not optimally labeled with SiNPs. At the composition of 50%:50%, NSCs were labeled with much more NP-PEG11-3400. It appears that the shorter functional Mal-PEG3400 works better than the Mal-PEG5000. To ensure the reactivity between maleimide-thiol covalent bond formation, all future cell labeling steps were done at 37° C. At least 50% of functional Mal-PEGs in the coating to ensure NSC labeling is required. It appears that the short Mal-PEG3400 works better than the longer Mal-PEG5000, so we then tested Mal-PEG2000. It appears that labeling NSCs via functionalized PEGylated SiNPs reaches a limit which is consistent with what we found in the literature. NP attachment to the cell surface will eventually plateau as more NPs conjugate to cells. At 50%:50% composition, there was not much difference in the size of functional Mal-PEGs while making functional SiNPs. Mal-PEG2000 and Mal-PEG3400 appear to work better than Mal-PEG5000. At 80%:20%, Mal-PEG2000 works much better than Mal-PEG3400 and Mal-PEG5000 as it was able to label NSCs while the other two could not.

Plain fluorescent silica particles are produced by hydrolysis of orthosilicates and related compounds. They have a hydrophilic surface with terminal Si—OH-groups. The fluorescent silica particles are monodisperse and nonporous in the size range of 10 nm to 1.5 μm with a density of 2.0 g/cm³. Red-Hydroxyl NP: we used the 500 nm plain surface (i.e., unmodified silica nanoparticle), non-porous, spherical SiNP, with a density of 2.0 g/cm³ and Ex: 569 nm Em: 585 nm. (negative charge) Green-Hydroxyl NP: we used the 500 nm plain surface (i.e., unmodified silica nanoparticle), non-porous, spherical SiNP, with a density of 2.0 g/cm³ and Ex: 485 nm Em: 510 nm. (negative charge) Amine-NP: for the human tissues experiment we used the 500 nm NH2-surface, non-porous, spherical SiNP, with a density of 2.0 g/cm³ and Ex: 569 nm Em: 585 nm. (positive charge).

For Iron NPs: We used a custom made iron oxide core coated in a red fluorescent silica shell, with an average diameter of about 500 nm, plain surface, non-porous, spherical SiNP and Ex: 569 nm Em: 585 nm. (Negative charge). The particles are produced by hydrolysis of orthosilicates in the presence of magnetite and show a homogeneous distribution of magnetite in the silica matrix by the special preparation method. The plain particles have a hydrophilic surface with terminal Si—OH-bonds.

Protocols on how to make dye doped fluorescent silica NPs (the most common one) in microemulsion method and covalently attached fluorophores silica NPs, both in Stober and microemulsion methods. To make doped fluorescent silica NPs in microemulsion method: Briefly, 1.8 mL of Triton X-100, 7.5 mL of cyclohexane, and 1.6 mL of n-hexanol were mixed, and an appropriate amount of ultrapure water was added to form a transparent microemulsion. The dye mixture solution was then added into the microemulsion and stirred for 30 min. TEOS was added as a precursor for silica formation and hydrolyzed under the catalysis of ammonia (volume ratio of TEOS to ammonia was 1.7). The reaction proceeded over a period of 24 h at room temperature. After the reaction was completed, the nanoparticles were precipitated by addition of ethanol/isopropanol and were washed with ethanol and water, respectively, for several times to remove the surfactant and excess dye molecules from the particles.

Additionally, we have synthesized dye doped fluorescent silica NPs with the addition of 3-Aminopropyltriethoxysilane (APTS); resulting in NPs with different terminal moieties (e.g., NH₂).

B. Tumor Detection by Fluorescent Nanoparticles

In this study using a metastatic mouse model of ovarian cancer, we have shown that when the red-fluorescently-labeled silica nanoparticles are administrated intraperitoneally (IP), they can selectively and sensitively detect ovarian tumor metastases, while not targeting other healthy tissues, hence showing selective tumor targeting. For these studies, the nanoparticles were injected IP and after 4 days, the animals were euthanized, the organs in the IP cavity were removed and a fluorescent whole-body imaging system was used to demonstrate that nanoparticles were selectively localized at tumor sites. Tumors and adjacent healthy tissue were then removed and prepared for confocal imaging which confirmed that nanoparticles only bound to cancer tissue.

The experimental setup is as follows: two types of mice were used: Nude and SCID; Tumor: OVCAR8.eGFP (Green) (Injected: Day 0); Treatment: NP only (NP: Red) (Injected: Day 21); Imaging+Harvest (Day 25); Imaging by a whole body imaging system (Ami-X), and by sectioning and staining the tumors and organs and image them by a confocal.

Ovarian cancer is a deadly disease that afflicts approximately 22,000 women per year in the US. Once it has reached stage III and metastasized to the abdominal cavity, there is a 5-year survival rate of only 34%. Surgery is the frontline therapy for this disease and has two purposes. The first is to stage the cancer to see how far the cancer has spread from the ovary. The second is to remove as much of the disease as possible this is called debulking. Surgery is critical to patient outcomes with survival linked to the degree of tumor removed from the abdomen. The current clinical standard is to remove all tumors larger than 1 cm in diameter, as this is roughly the limit of detection by eye. Despite achieving no gross visible disease at the end of surgery, 50-70% of patients will relapse. Therefore, there is a need for better detection techniques during surgery, to enable surgeons to detect smaller tumors and remove them. In the present example utilize NIR-fluorescently-labeled silica nanoparticles to selectively and sensitively detect small ovarian tumors in the abdominal cavity and by that improving surgery outcome.

Fluorescently-labeled silica nanoparticles are administrated IP, can selectively and sensitively detect ovarian tumor metastases, while not targeting other healthy tissues, hence showing selective tumor targeting. For these studies, the nanoparticles were injected IP and after 4 days, the animals were euthanized, the organs in the IP cavity were removed and a fluorescent whole-body imaging system was used to demonstrate that nanoparticles were selectively localized at tumor sites. Tumors and adjacent healthy tissue were then removed and prepared for confocal imaging which confirmed that nanoparticles only bound to cancer tissue.

We have tested the detection of the silica NP on different types of ovarian cancers. The hydroxyl-silica nanoparticles (e.g., unmodified) demonstrated good correlation in coverage with OVCAR-8, and SKOV-3 ovarian tumors. In some cases, cells with red-fluorescent silica nanoparticles stained positive for CD45, CD11b and f4/80 markers which are common for macrophages.

1. A method of detecting a cancer cell or tumor in a subject comprising:
a) administering into the peritoneum of said subject a nanoparticle, wherein the nanoparticle comprises a detectable agent within the silica nanoparticle and wherein the nanoparticle is an unmodified silica nanoparticle comprising substantially only terminal hydroxyl moieties at the nanoparticle surface; and
b) detecting said nanoparticle at the site of said cancer cell or said tumor in said subject;
thereby detecting the cancer cell or tumor in said subject.

2-10. (canceled)

11. The method of claim 1, wherein the detectable agent is a radioisotope, fluorophore, electron-dense reagent, enzyme, biotin, paramagnetic agent, or magnetic agent.

12. The method of claim 1, wherein the detectable agent is a fluorophore.

13. The method of claim 1, wherein the detectable agent is a fluorophore having a maximum emission wavelength from about 495 nm to about 570 nm.

14. The method of claim 1, wherein the detectable agent is a fluorophore having a maximum emission wavelength from about 570 nm to about 620 nm.

15. The method of claim 1, wherein the detectable agent is a fluorophore having a maximum emission wavelength from about 620 nm to about 650 nm.

16. The method of claim 1, wherein the detectable agent is a fluorophore having a maximum emission wavelength from about 710 nm to about 850 nm.

17. The method of claim 1, wherein the detectable agent is a fluorophore having a maximum emission wavelength from about 850 nm to about 1350 nm.

18. The method of claim 1, wherein the detectable agent is cyanine, heptamethine, xanthene, rhodamine, fluorescein, boron-dipyrromethene, boron dipyridyl, naphthalene, coumarin, acridine, acridinium, tetrapyrrole, tetraphenylethene, oxazine, pyrene, oxadiazole, subphthalocyanine, carbopyrinin, benzopyrinium, or phthalocyanine.

19. The method of claim 1, wherein the average longest dimension of the nanoparticle is from about 10 nm to about 1000 nm.

20. The method of claim 1, wherein the average longest dimension of the nanoparticle is from about 10 nm to about 600 nm.

21. The method of claim 1, wherein the average longest dimension of the nanoparticle is from about 100 nm to about 400 nm.

22. The method of claim 1, wherein the average longest dimension of the nanoparticle is from about 170 nm to 270 nm.

23-26. (canceled)

27. The method of claim 1, wherein the tumor is an ovarian tumor, bladder tumor, pancreatic tumor, colorectal tumor, gastric tumor, bone tumor, spinal tumor, or liver tumor.

28-30. (canceled)
